SEQUENCE LISTING (9 sequences)

SEQ ID NO: 1 - DNA, 1249 bp, Homo sapiens
  CDS (102)...(1169)
acagtaacctaaggaattttctttgaaattcagtgctgccatttgtttgcttatggagtggcacagagcccacagcatttggctctgcctcagaggcaaacatggctacagcagccaaagagaaacacaaaggaggacactctaggaatggaacctataagtggggacttctgggtgcacgaattgtgataaagaagcagtgctttccggtgatagctaggtgcaataaaaccaaacaagtagattccatacctcatcgtgcatatgcaaagcctagcccattctttcctgcagtagttgtaactagtgaatcaaaggatccccgagttaagcaaaatccaactcatccactcctagctgaaggttctaatgctgctcatttccataaaacaaagatttcccagagcctgaagtggcttcctgttggctacttaaacagcacaacatatgagagcctctggggacatactgctcagacatctgccacaatatgtgtcaccccctcactgggctcccccagtgtaaaaggacaggcactgcaactcctgtcccatccatacttaggggcattgccagaggccaggaccagaggactttggacacgactccacgtacggcatctcatgcaggtgccacagcagccctgcatagcatttgctgtatcatccccattgcaccagggagggaaggaaacagaggatgcctggtcttttcatataccatccacgatacaccgatggcccacctctccttctgtccagctctcaagcctaagggccagcacacagctcctgtgtctgcacatcaaccttgcaaagtgcttcatctggggaattcttgcacaggttctgtcttgtcatcatcaccgcattacgcagtttgtgggcatgttgacttttgtgtgcgctggtgattccagcaagctttcccctgatatacgaacactggtcagacgtctgacttcatatctggtgtctgcatcagtgcttctaaaaagcaagcagatggctctttgctctgcttccaggatcctggatgtctcagtctccccaggtgatcatgtggcctcagaagccagagctagatgtgctcctgatcttgtagcccctcgtcctatgcctcatcaaaaccaggggtcattggaatggactgaagaaggagaaaactagaaaggatctgagggaagtggtctggctgaaagcttgacgtggacagagtcctggctctcaggcagacacttaaattctcc

SEQ ID NO: 2 - protein, 355 aa, Homo sapiens
MATAAKEKHKGGHSRNGTYKWGLLGARIVIKKQCFPVIARCNKTKQVDSIPHRAYAKPSPFFPAVVVTSESKDPRVKQNPTHPLLAEGSNAAHFHKTKISQSLKWLPVGYLNSTTYESLWGHTAQTSATICVTPSLGSPSVKGQALQLLSHPYLGALPEARTRGLWTRLHVRHLMQVPQQPCIAFAVSSPLHQGGKETEDAWSFHIPSTIHRWPTSPSVQLSSLRASTQLLCLHINLAKCFIWGILAQVLSCHHHRITQFVGMLTFVCAGDSSKLSPDIRTLVRRLTSYLVSASVLLKSKQMALCSASRILDVSVSPGDHVASEARARCAPDLVAPRPMPHQNQGSLEWTEEGEN

SEQ ID NO: 3 - DNA, 1068 bp, Homo sapiens
atggctacagcagccaaagagaaacacaaaggaggacactctaggaatggaacctataagtggggacttctgggtgcacgaattgtgataaagaagcagtgctttccggtgatagctaggtgcaataaaaccaaacaagtagattccatacctcatcgtgcatatgcaaagcctagcccattctttcctgcagtagttgtaactagtgaatcaaaggatccccgagttaagcaaaatccaactcatccactcctagctgaaggttctaatgctgctcatttccataaaacaaagatttcccagagcctgaagtggcttcctgttggctacttaaacagcacaacatatgagagcctctggggacatactgctcagacatctgccacaatatgtgtcaccccctcactgggctcccccagtgtaaaaggacaggcactgcaactcctgtcccatccatacttaggggcattgccagaggccaggaccagaggactttggacacgactccacgtacggcatctcatgcaggtgccacagcagccctgcatagcatttgctgtatcatccccattgcaccagggagggaaggaaacagaggatgcctggtcttttcatataccatccacgatacaccgatggcccacctctccttctgtccagctctcaagcctaagggccagcacacagctcctgtgtctgcacatcaaccttgcaaagtgcttcatctggggaattcttgcacaggttctgtcttgtcatcatcaccgcattacgcagtttgtgggcatgttgacttttgtgtgcgctggtgattccagcaagctttcccctgatatacgaacactggtcagacgtctgacttcatatctggtgtctgcatcagtgcttctaaaaagcaagcagatggctctttgctctgcttccaggatcctggatgtctcagtctccccaggtgatcatgtggcctcagaagccagagctagatgtgctcctgatcttgtagcccctcgtcctatgcctcatcaaaaccaggggtcattggaatggactgaagaaggagaaaactag

SEQ ID NO: 4 - DNA, 1704 bp, Homo sapiens
  CDS (1)...(1704)
atggctacagcagccaaagagaaacacaaaggaggacactctaggaatggaacctataagtggggacttctgggtgcacgaattgtgataaagaagcagtgctttccggtgatagctaggtgcaataaaaccaaacaagtagattccatacctcatcgtgcatatgcaaagcctagcccattctttcctgcagtagttgtaactagtgaatcaaaggatccccgagttaagcaaaatccaactcatccactcctagctgaaggttctaatgctgctcatttccataaaacaaagatttcccagagcctgaagtggcttcctgttggctacttaaacagcacaacatatgagagcctctggggacatactgctcagacatctgccacaatatgtgtcaccccctcactgggctcccccagtgtaaaaggacaggcactgcaactcctgtcccatccatacttaggggcattgccagaggccaggaccagaggactttggacacgactccacgtacggcatctcatgcaggtgccacagcagccctgcatagcatttgctgtatcatccccattgcaccagggagggaaggaaacagaggatgcctggtcttttcatataccatccacgatacaccgatggcccacctctccttctgtccagctctcaagcctaagggccagcacacagctcctgtgtctgcacatcaaccttgcaaagtgcttcatctggggaattcttgcacaggttctgtcttgtcatcatcaccgcattacgcagtttgtgggcatgttgacttttgtgtgcgctggtgattccagcaagctttcccctgatatacgaacactggtcagacgtctgacttcatatctggtgtctgcatcagtgcttctaaaaagcaagcagacattgcaagcacattatgtcacttctgagacccagatgcgcctgggctcctcctttaccaagccaaacattcctgtgaacaggcttgagtggaagccctggggtgggacgacacaaaggcaggcatctccgttccaactagatggaacccagccccacttgctttccaatgaccttacctactttgcatactttgtcttcactggggcgaaacgtcagcactggttaaccagaaccatcaaccccatcagacctgaggagcaggtgctgagggacagcaacgtcacagccaggaagacccttccgaaatctccaccctttgctgtgggttttggccacacccagggtggagctaaggagggtggggcagcaggattctcacataacttggaactttgtgatgatctggaatcagccctctctgaagcaagttctttcctccctgcagctctcttgggtgggatatcaaatgaccccagccagctttctcagtggagccgcccaaatcaggtccttgggaaacactcccacgttccctttgcccctcaagcctacaccagctacccgttgccccgggagccagaggctgctgctgtggccagcatggacagcactaaaccaaagccgcctctgttctccactgctggcaggaccaagctgagaccaggaccaaaactgtgggatgtgttggccacatacttccagccccacagcacacagcaggcatggagggcgggaccctgtcaacagtggctgaaaccagaaatgtacctgaaagggcagcttctagtagaactgtaa

SEQ ID NO: 5 - protein, 567 aa, Homo sapiens [sequence omitted]

SEQ ID NO: 6 - DNA, 24 bp, Artificial Sequence
  random primer
tcaactgactagatgtacatggac

SEQ ID NO: 7 - DNA, 20 bp, Artificial Sequence
  Primer1
tctcagtctccccaggtgat

SEQ ID NO: 8 - DNA, 20 bp, Artificial Sequence
  Primer2
gggctacaagatcaggagca

SEQ ID NO: 9 - DNA, 24 bp, Artificial Sequence
  Probe1
catgtggcctcagaagccagagct

(19) United States
(12) Patent Application Publication
(10) Pub. No.: US 2005/0255545 A1
(43) Pub. Date: Nov. 17, 2005

(54) Regulation of human hematopoietin receptor-like protein

(76) Inventor: Timothy J. Smith, Cambridge, MA (US)

Correspondence Address: BANNER & WITCOFF, 1001 G STREET N W, SUITE 1100, WASHINGTON, DC, 20001, US

(21) Appl. No.: 10/503,051

(22) PCT Filed: Jan. 31, 2003
(86) PCT No.: PCT/EP03/00964

§ 371 Date: Jul. 8, 2005

(30) Foreign Application Priority Data
Feb. 1, 2002 (US) 60352866
Jul. 31, 2002 (US) 60399382

(51) Int. Cl. C12Q 1/68; C07H 21/04; C12P 21/06; C07K 14/705; C12N 15/09

(52) U.S. Cl. 435/69.1; 435/6; 435/320.1; 435/325; 530/350; 536/23.5

(57) ABSTRACT

Reagents that regulate human hematopoietin receptor-like protein and reagents which bind to human hematopoietin receptor-like gene products can play a role in preventing, ameliorating, or correcting dysfunctions or diseases including, but not limited to, CNS disorders, COPD, cardiovascular disorders, liver disorders, cancer, asthma, and hematological disorders.

This application incorporates by reference co-pending U.S. provisional applications Ser. No. 60/352,866 filed Feb. 1, 2002 and Ser. No. 60/399,382 filed Jul. 31, 2002.

### FIELD OF THE INVENTION

The invention relates to the regulation of human hematopoietin receptor-like protein.

### BACKGROUND OF THE INVENTION

A variety of diseases, including malignancy and immunodeficiency, are related to malfunction within the lympho-hematopoietic system. U.S. Pat. No. 5,643,748. Some of these conditions could be alleviated and/or cured by repopulating the hematopoietic system with progenitor cells, which when triggered to differentiate would overcome the patient's deficiency. Therefore, the ability to initiate and regulate hematopoiesis is of great importance (McCune et al., 1988, Science 241:1632).

The process of blood cell formation, by which a small number of self-renewing stem cells give rise to lineage specific progenitor cells that subsequently undergo proliferation and differentiation to produce the mature circulating blood cells has been shown to be at least in part regulated by specific hormones. These hormones are collectively known as hematopoietic growth factors or cytokines (Metcalf, 1985, Science 229:16; Dexter, 1987, J. Cell Sci. 88:1; Golde and Gasson, 1988, Scientific American, July:62; Tabbara and Robinson, 1991, Anti-Cancer Res. 11:81; Ogawa, 1989, Environ. Health Presp. 80:199; Dexter, 1989, Br. Med. Bull. 45:337).

With the advent of recombinant DNA technology, the genes encoding a number of these molecules have now been molecularly cloned and expressed in recombinant form (Souza et al., 1986, Science 232:61; Gough et al., 1984, Nature 309:763; Yokota et al., 1984, Proc. Natl. Acad. Sci. U.S.A. 81:1070; Kawasaki et al., 1985, Science 230:291). These cytokines have been studied in their structure, biology and even therapeutic potential. Some of the most well characterized factors include erythropoietin (EPO), stem cell factor (SCF), granulocyte macrophage colony stimulating factor (GM-CSF), macrophage colony stimulating factor (M-CSF), granulocyte colony stimulating factor (G-CSF), and the interleukins (IL-1 to IL-14).

These factors act on different cell types at different stages during blood cell development, and their potential uses in medicine are far-reaching which include blood transfusions, bone marrow transplantation, correcting immunosuppressive disorders, cancer therapy, wound healing, and activation of the immune response. (Golde and Gasson, 1988, Scientific American, July:62).

Apart from inducing proliferation and differentiation of hematopoietic progenitor cells, such cytokines have also been shown to activate a number of functions of mature blood cells (Stanley et al., 1976, J. Exp. Med. 143:631; Schrader et al., 1981, Proc. Natl. Acad. Sci. U.S.A. 78:323; Moore et al., 1980, J. Immunol. 125:1302; Kurland et al., 1979, Proc. Natl. Acad. Sci. U.S.A. 76:2326; Handman and Burgess, 1979, J. Immunol. 122:1134; Vadas et al., 1983, Blood 61:1232; Vadas et al., 1983, J. Immunol. 130:795), including influencing the migration of mature hematopoietic cells (Weibart et al., 1986, J. Immunol. 137:3584).

Cytokines exert their effects on target cells by binding to specific cell surface receptors. A number of cytokine receptors have been identified and the genes encoding them molecularly cloned. Several cytokine receptors have recently been classified into a hematopoietin receptor (HR) superfamily. The grouping of these receptors was based on the conservation of key amino acid motifs in the extracellular domains (Bazan, 1990, Immunology Today 11:350) (FIG. 1). The HR family is defined by three conserved motifs in the extracellular domain of these receptors. The first is a Trp-Ser-X-Trp-Ser (WSXWS box) motif (SEQ ID NO: 2) which is highly conserved and located amino-terminal to the transmembrane domain. Most members of the HR family contain this motif. The second consists of four conserved cysteine residues located in the N-terminal half of the extracellular region. The third is a conserved fibronectin Type III (FN III) domain which is located between the WSXWS box and the cysteines. The members of the HR family include receptors for ligands such as erythropoietin (EPO), granulocyte colony stimulating factor (G-CSF) (Fukunaga, 1990, Cell 61:341), granulocyte-macrophage colony stimulating factor (GM-CSF), interleukin-3 (IL-3), IL-4, IL-5, IL-6, IL-7, and IL-2 (.beta.-subunit) (Cosman, 1990, TIBS 15:265).

Ligands for the HR are critically involved in the maturation and differentiation of blood cells. For example, IL-3 promotes the proliferation of early multilineage pluripotent stem cells, and synergizes with EPO to produce red cells. IL-6 and IL-3 synergize to induce proliferation of early hematopoietic precursors. GM-CSF has been shown to induce the proliferation of granulocytes as well as increase macrophage function. IL-7 is a bone marrow-derived cytokine that plays a role in producing immature T and B lymphocytes. IL-4 induces proliferation of antigen-primed B cells and antigen-specific T cells. Thus, members of this receptor superfamily are involved in the regulation of the hematopoietic system.

There is a need in the art to identify additional hematopoietin receptors, which can be regulated to provide therapeutic effects.

### BRIEF SUMMARY OF THE INVENTION

It is an object of the invention to provide reagents and methods of regulating a human hematopoietin receptor-like protein. This and other objects of the invention are provided by one or more of the embodiments described below.

One embodiment of the invention is a hematopoietin receptor-like protein polypeptide comprising an amino acid sequence selected from the group consisting of:

amino acid sequences which are at least about 88% identical to

the amino acid sequence shown in SEQ ID NO: 2;
the amino acid sequence shown in SEQ ID NO: 2;
   amino acid sequences which are at least about 88% identical to
the amino acid sequence shown in SEQ ID NO: 5; and
the amino acid sequence shown in SEQ ID NO: 5.

Yet another embodiment of the invention is a method of screening for agents which decrease extracellular matrix degradation. A test compound is contacted with a hematopoietin receptor-like protein polypeptide comprising an amino acid sequence selected from the group consisting of:

amino acid sequences which are at least about 88% identical to

the amino acid sequence shown in SEQ ID NO: 2;
the amino acid sequence shown in SEQ ID NO: 2;
   amino acid sequences which are at least about 88% identical to,
the amino acid sequence shown in SEQ ID NO: 5; and
the amino acid sequence shown in SEQ ID NO: 5.

Binding between the test compound and the hematopoietin receptor-like protein polypeptide is detected. A test compound which binds to the hematopoietin receptor-like protein polypeptide is thereby identified as a potential agent for decreasing extracellular matrix degradation. The agent can work by decreasing the activity of the hematopoietin receptor-like protein.

Another embodiment of the invention is a method of screening for agents which decrease extracellular matrix degradation. A test compound is contacted with a polynucleotide encoding a hematopoietin receptor-like protein pplypeptide, wherein the polynucleotide comprises a nucleotide sequence selected from the group consisting of:

nucleotide sequences which are at least about 50% identical to

the nucleotide sequence shown in SEQ ID NO: 1;
the nucleotide sequence shown in SEQ ID NO: 1;
   nucleotide sequences which are at least about 50% identical to
the nucleotide sequence shown in SEQ ID NO: 3;
the nucleotide sequence shown in SEQ ID NO: 3;
   nucleotide sequences which are at least about 50% identical to
the nucleotide sequence shown in SEQ ID NO: 4; and
the nucleotide sequence shown in SEQ ID NO: 4.

Binding of the test compound to the polynucleotide is detected. A test compound which binds to the polynucleotide is identified as a potential agent for decreasing extracellular matrix degradation. The agent can work by decreasing the amount of the hematopoietin receptor-like protein through interacting with the hematopoietin receptor-like protein mRNA.

Another embodiment of the invention is a method of screening for agents which regulate extracellular matrix degradation. A test compound is contacted with a hematopoietin receptor-like protein polypeptide comprising an amino acid sequence selected from the group consisting of:

amino acid sequences which are at least about 88% identical to

the amino acid sequence shown in SEQ ID NO: 2;
the amino acid sequence shown in SEQ ID NO: 2;
   amino acid sequences which are at least about 88% identical to
the amino acid sequence shown in SEQ ID NO: 5; and
the amino acid sequence shown in SEQ ID NO: 5.

A hematopoietin receptor-like protein activity of the polypeptide is detected. A test compound which increases hematopoietin receptor-like protein activity of the polypeptide relative to hematopoietin receptor-like protein activity in the absence of the test compound is thereby identified as a potential agent for increasing extra-cellular matrix degradation. A test compound which decreases hematopoietin receptor-like protein activity of the polypeptide relative to hematopoietin receptor-like protein activity in the absence of the test compound is thereby identified as a potential agent for decreasing extracellular matrix degradation.

Even another embodiment of the invention is a method of screening for agents which decrease extracellular matrix degradation. A test compound is contacted with a hematopoietin receptor-like protein product of a polynucleotide which comprises a nucleotide sequence selected from the group consisting of:

nucleotide sequences which are at least about 50% identical to

the nucleotide sequence shown in SEQ ID NO: 1;
the nucleotide sequence shown in SEQ ID NO: 1;
   nucleotide sequences which are at least about 50% identical to
the nucleotide sequence shown in SEQ ID NO: 3;
the nucleotide sequence shown in SEQ ID NO: 3;
   nucleotide sequences which are at least about 50% identical to
the nucleotide sequence shown in SEQ ID NO: 4; and
the nucleotide sequence shown in SEQ ID NO: 4.

Binding of the test compound to the hematopoietin receptor-like protein product is detected. A test compound which binds to the hematopoietin receptor-like protein product is thereby identified as a potential agent for decreasing extracellular matrix degradation.

Still another embodiment of the invention is a method of reducing extracellular matrix degradation. A cell is contacted with a reagent which specifically binds to a polynucleotide encoding a hematopoietin receptor-like protein polypeptide or the product encoded by the polynucleotide, wherein the polynucleotide comprises a nucleotide sequence selected from the group consisting of:

nucleotide sequences which are at least about 50% identical to.

the nucleotide sequence shown in SEQ ID NO: 1;
the nucleotide sequence shown in SEQ ID NO: 1;
   nucleotide sequences which are at least about 50% identical to
the nucleotide sequence shown in SEQ ID NO: 3;
the nucleotide sequence shown in SEQ ID NO: 3;
   nucleotide sequences which are at least about 50% identical to
the nucleotide sequence shown in SEQ ID NO: 4; and
the nucleotide sequence shown in SEQ ID NO: 4.

Hematopoietin receptor-like protein activity in the cell is thereby decreased.

The invention thus provides a human hematopoietin receptor-like protein that can be used to identify test compounds that may act, for example, as activators or inhibitors. Human hematopoietin receptor-like protein and fragments thereof also are useful in raising specific antibodies that can block the protein and effectively reduce its activity.

### DETAILED DESCRIPTION OF THE INVENTION

The invention relates to an isolated polynucleotide from the group consisting of:

a) a polynucleotide encoding a hematopoietin receptor-like protein polypeptide comprising an amino acid sequence selected from the group consisting of:

amino acid sequences which are at least about 88% identical to

the amino acid sequence shown in SEQ ID NO: 2;
the amino acid sequence shown in SEQ ID NO: 2;
   amino acid sequences which are at least about 88% identical to
the amino acid sequence shown in SEQ ID NO: 5; and
the amino acid sequence shown in SEQ ID NO: 5.
   b) a polynucleotide comprising the sequence of SEQ ID NO: 1, 3 or 4;
   c) a polynucleotide which hybridizes under stringent conditions to a polynucleotide specified in (a) and (b) and encodes a hematopoietin receptor-like protein polypeptide;
   d) a polynucleotide the sequence of which deviates from the polynucleotide sequences specified in (a) to (c) due to the degeneration of the genetic code and encodes a hematopoietin receptor-like protein polypeptide; and
   e) a polynucleotide which represents a fragment, derivative or allelic variation of a polynucleotide sequence specified in (a) to (d) and encodes a hematopoietin receptor-like protein polypeptide.

Furthermore, it has been discovered by the present applicant that a novel hematopoietin receptor-like protein, particularly a human hematopoietin receptor-like protein, can be used in therapeutic methods to treat CNS disorders, COPD, cardiovascular disorders, liver disorders, cancer, asthma and hematological disorders. Human hematopoietin receptor-like protein comprises the amino acid sequence shown in SEQ ID NO: 2 or 5. Coding sequence for human hematopoietin receptor-like protein are shown in SEQ ID NO: 3 and 4. The ORF represented by SEQ ID NO: 3 is contained within the longer sequence shown in SEQ ID NO: 1. This sequence is located on chromosome 18.

Human hematopoietin receptor-like protein also can be used to screen for human hematopoietin receptor-like protein activators and inhibitors.

Polypeptides

Human hematopoietin receptor-like polypeptides according to the invention comprise at least 6, 10, 15, 20, 25, 50, 75, 100, 125, 150, 175, 200, 225, 250, 275, 300, 325, 350, or 355 contiguous amino acids selected from the amino acid sequence shown in SEQ ID NO: 2 or at least 6, 10, 15, 20, 25, 50, 75, 100, 125, 150, 175, 200, 225, 250, 275, 300, 325, 350, 375, 400, 450, 500, 550, or 567 contiguous amino acids selected from the amino acid sequence shown in SEQ ID NO: 5 or a biologically active variant thereof, as defined below. A hematopoietin receptor-like polypeptide of the invention therefore can be a portion of a hematopoietin receptor-like protein, a full-length hematopoietin receptor-like protein, or a fusion protein comprising all or a portion of a hematopoietin receptor-like protein.

Biologically Active Variants

Human hematopoietin receptor-like polypeptide variants which are biologically active, e.g., retain a functional activity, also are human hematopoietin receptor-like polypeptides. Preferably, naturally or non-naturally occurring human hematopoietin receptor-like polypeptide variants have amino acid sequences which are at least about 88, 90, 95, 96, 97, 98, or 99% identical to the amino acid sequence shown in SEQ ID NO: 2 or 5 or a fragment thereof. Percent identity between a putative human hematopoietin receptor-like polypeptide variant and an amino acid sequence of SEQ ID NO: 2 or 5 is determined by conventional methods. See, for example, Altschul et al., Bull Math. Bio. 48:603 (1986), and Henikoff & Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff & Henikoff, 1992.

Those skilled in the art appreciate that there are many established algorithms available to align two amino acid sequences. The “FASTA” similarity search algorithm of Pearson & Lipman is a suitable protein alignment method for examining the level of identity shared by an amino acid sequence disclosed herein and the amino acid sequence of a putative variant. The FASTA algorithm is described by Pearson & Lipman, Proc. Nat'l Acad. Sci. USA 85:2444(1988), and by Pearson, Meth. Enzymol. 183:63 (1990). Briefly, FASTA first characterizes sequence similarity by identifying regions shared by the query sequence (e.g., SEQ ID NO: 2 or 5) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman & Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a human hematopoietin receptor-like polypeptide can be found using computer programs well known in the art, such as DNASTAR software.

The invention additionally, encompasses hematopoietin receptor-like polypeptides that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications can be carried out by known techniques including, but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The hematopoietin receptor-like polypeptides may also be modified with a detectable label, such as an enzymatic, fluorescent, isotopic or affinity label to allow for detection and isolation of the protein.

The invention also provides chemically modified derivatives of hematopoietin receptor-like polypeptides that may provide additional advantages such as increased solubility, stability and circulating time of the polypeptide, or decreased immunogenicity (see U.S. Pat. No. 4,179,337). The chemical moieties for derivitization can be selected from water soluble polymers such as polyethylene glycol, ethylene glycol/propylene glycol copolymers, carboxymethylcellulose, dextran, polyvinyl alcohol, and the like. The polypeptides can be modified at random or predetermined positions within the molecule and can include one, two, three, or more attached chemical moieties.

Whether an amino acid change or a polypeptide modification results in a biologically active hematopoietin receptor-like polypeptide can readily be determined by assaying for functional activity of the receptor.

Fusion Proteins

Fusion proteins are useful for generating antibodies against hematopoietin receptor-like polypeptide amino acid sequences and for use in various assay systems. For example, fusion proteins can be used to identify proteins that interact with portions of a human hematopoietin receptor-like polypeptide. Protein affinity chromatography or library-based assays for protein-protein interactions, such as the yeast two-hybrid or phage display systems, can be used for this purpose. Such methods are well known in the art and also can be used as drug screens.

A human hematopoietin receptor-like polypeptide fusion protein comprises two polypeptide segments fused together by means of a peptide bond. The first polypeptide segment comprises at least 6, 10, 15, 20, 25, 50, 75, 100, 125, 150, 175, 200, 225, 250, 275, 300, 325, 350, or 355 contiguous amino acids of SEQ ID NO: 2 or at least 6, 10, 15, 20, 25, 50, 75, 100, 125, 150, 175, 200, 225, 250, 275, 300, 325, 350, 375, 400, 450, 500, 550, or 567 contiguous amino acids selected from the amino acid sequence shown in SEQ ID NO: 5 or of a biologically active variant, such as those described above. The first polypeptide segment also can comprise full-length hematopoietin receptor-like protein.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the hematopoietin receptor-like polypeptide-encoding sequence and the heterologous protein sequence, so that the hematopoietin receptor-like polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from SEQ ID NO: 1 in proper reading frame with nucleotides encoding the second polypeptide segment and expressing the DNA construct in a host cell, as is known in the art. Many kits for constructing fusion proteins are available from companies such as Promega Corporation (Madison, WI), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human hematopoietin receptor-like polypeptide can be obtained using hematopoietin receptor-like polypeptide. polynucleotides (described below) to make suitable probes or primers for screening cDNA expression libraries from other species, such as mice, monkeys, or yeast, identifying cDNAs which encode homologs of hematopoietin receptor-like polypeptide, and expressing the cDNAs as is known in the art.

Polynucleotides

A human hematopoietin receptor-like polynucleotide can be single- or double-stranded and comprises a coding sequence or the complement of a coding sequence for a hematopoietin receptor-like polypeptide. A coding sequence for human hematopoietin receptor-like protein is shown in SEQ ID NO: 3 and 4.

Degenerate nucleotide sequences encoding human hematopoietin receptor-like polypeptides, as well as homologous nucleotide sequences which are at least about 50, 55, 60, 65, 70, preferably about 75, 90, 96, 98, or 99% identical to the nucleotide sequence shown in SEQ ID NO: 1, 3 or 4 or their complements also are hematopoietin receptor-like polynucleotides. Percent sequence identity between the sequences of two polynucleotides is determined using computer programs such as ALIGN which employ the FASTA algorithm, using an affine gap search with a gap open penalty of —12 and a gap extension penalty of —2. Complementary DNA (cDNA) molecules, species homologs, and variants of hematopoietin receptor-like polynucleotides that encode biologically active hematopoietin receptor-like polypeptides also are hematopoietin receptor-like polynucleotides. Polynucleotide fragments comprising at least 8, 9, 10, 11, 12, 15, 20, or 25 contiguous nucleotides of SEQ ID NO: 1, 3 or 4 or their complements also are hematopoietin receptor-like polynucleotides. These fragments can be used, for example, as hybridization probes or as antisense oligonucleotides.

Identification of Polynucleotide Variants and Homologs

Variants and homologs of the hematopoietin receptor-like polynucleotides described above also are hematopoietin receptor-like polynucleotides. Typically, homologous hematopoietin receptor-like polynucleotide sequences can be identified by hybridization of candidate polynucleotides to known hematopoietin receptor-like polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each—homologous sequences can be identified which contain at most about 25-30% basepair mismatches. More preferably, homologous nucleic acid strands contain 15-25% basepair mismatches, even more preferably 5-15% basepair mismatches.

Species homologs of the hematopoietin receptor-like polynucleotides disclosed herein also can be identified by making suitable probes or primers and screening cDNA expression libraries from other species, such as mice, monkeys, or yeast. Human variants of hematopoietin receptor-like polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human hematopoietin receptor-like polynucleotides or hematopoietin receptor-like polynucleotides of other species can therefore be identified by hybridizing a putative homologous hematopoietin receptor-like polynucleotide with a polynucleotide having a nucleotide sequence of SEQ ID NO: 1, 3 or 4 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to hematopoietin receptor-like polynucleotides or their complements following stringent hybridization and/or wash conditions also are hematopoietin receptor-like polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a hematopoietin receptor-like polynucleotide having a nucleotide sequence shown in SEQ ID NO: 1, 3 or 4 or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):
Tₘ=81.5° C.−16.6(log₁₀[Na⁺])+0.41 (% G+C)−0.63 (% formamide)−600/l),
where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A human hematopoietin receptor-like polynucleotide can be isolated free of other cellular components such as membrane components, proteins, and lipids. Polynucleotides can be made by a cell and isolated using standard nucleic acid purification techniques, or synthesized using an amplification technique, such as the polymerase. chain reaction (PCR), or by using an automatic synthesizer. Methods for isolating polynucleotides are routine and are known in the art. Any such technique for obtaining a polynucleotide can be used to obtain isolated hematopoietin receptor-like polynucleotides. For example, restriction enzymes and probes can be used to isolate polynucleotide fragments, which comprise hematopoietin receptor-like protein nucleotide sequences. Isolated polynucleotides are in preparations that are free or at least 70, 80, or 90% free of other molecules.

Human hematopoietin receptor-like cDNA molecules can be made with standard molecular biology techniques, using hematopoietin receptor-like mRNA as a template. Human hematopoietin receptor-like cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesize hematopoietin receptor-like polynucleotides. The degeneracy of the genetic code allows alternate nucleotide sequences to be synthesized which will encode a human hematopoietin receptor-like polypeptide having, for example, an amino acid sequence shown in SEQ ID NO: 2 or 5 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus. Sarkar, PCR Methods Applic. 2, 318-322, 1993; Triglia et al., Nucleic Acids Res. 16, 8186, 1988; Lagerstrom et al., PCR Methods Applic. 1, 111-119, 1991; Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif). See WO 01/98340

Obtaining Polynucleotides

Human hematopoietin receptor-like protein polypeptides can be obtained, for example, by purification from human cells, by expression of hematopoietin receptor-like protein polynucleotides, or by direct chemical synthesis.

Protein Purification

Human hematopoietin receptor-like protein polypeptides can be purified from any human cell which expresses the receptor, including host cells which have been transfected with hematopoietin receptor-like protein polynucleotides. A purified hematopoietin receptor-like protein polypeptide is separated from other compounds that normally associate with the hematopoietin receptor-like protein polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis.

A preparation of purified hematopoietin receptor-like protein polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a human hematopoietin receptor-like protein polynucleotide, the polynucleotide can be inserted into an expression vector which contains the necessary elements for the transcription and translation of the inserted coding sequence. Methods which are well known to those skilled in the art can be used to construct expression vectors containing sequences encoding hematopoietin receptor-like protein polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a human hematopoietin receptor-like protein polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems. See WO 01/98340.

Host Cells

A host cell strain can be chosen for its ability to modulate the expression of the inserted sequences or to process the expressed hematopoietin receptor-like protein polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells that have specific cellular machinery and characteristic mechanisms for post-translational activities (e.g., CHO, HeLa, MDCK, HEK293, and WI38) are available from the American Type Culture Collection (ATCC; 10801 University Boulevard, Manassas, Va. 20110-2209) and can be chosen to ensure the correct modification and processing of the foreign protein. See WO 01/98340.

Alternatively, host cells which contain a human hematopoietin receptor-like protein polynucleotide and which express a human hematopoietin receptor-like protein polypeptide can be identified by a variety of procedures known to those of skill in the art. Examples include enzyme-linked immunosorbent assay (ELISA), radioimmunoassay (RIA), and fluorescence activated cell sorting (FACS). Hampton et al., SEROLOGICAL METHODS: A LABORATORY MANUAL, APS Press, St. Paul, Minn., 1990) and Maddox et al., J. Exp. Med. 158, 1211-1216, 1983). See WO 01/98340.

A wide variety of labels and conjugation techniques are known by those skilled in the art and can be used in various nucleic acid and amino acid assays. Means for producing labeled hybridization or PCR probes for detecting sequences related to polynucleotides encoding hematopoietin receptor-like protein polypeptides include oligolabeling, nick translation, end-labeling, or PCR amplification using a labeled nucleotide. Alternatively, sequences encoding a human hematopoietin receptor-like protein polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a human hematopoietin receptor-like protein polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode hematopoietin receptor-like protein polypeptides can be designed to contain signal sequences which direct secretion of soluble hematopoietin receptor-like protein polypeptides through a prokaryotic or eukaryotic cell membrane or which direct the membrane insertion of membrane-bound hematopoietin receptor-like protein polypeptide. See WO 01/98340.

Chemical Synthesis

Sequences encoding a human hematopoietin receptor-like protein polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a human hematopoietin receptor-like protein polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of hematopoietin receptor-like protein polypeptides can be separately synthesized and combined using chemical methods to produce a full-length molecule. See WO 01/98340.

As will be understood by those of skill in the art, it may be advantageous to produce hematopoietin receptor-like protein polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter hematopoietin receptor-like protein polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a human hematopoietin receptor-like protein polypeptide. “Antibody” as used herein includes intact immunoglobulin molecules, as well as fragments thereof, such as Fab, F(ab′)₂, and Fv, which are capable of binding an epitope of a human hematopoietin receptor-like protein polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a human hematopoietin receptor-like protein polypeptide can be used therapeutically, as well as in immunochemical assays, such as Western blots, ELISAs, radioimmunoassays, immunohistochemical assays, immunoprecipitations, or other immunochernical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Typically, an antibody that specifically binds to a human hematopoietin receptor-like protein polypeptide provides a detection signal at least 5-, 10-, or 20-fold higher than a detection signal provided with other proteins when used in an immunochemical assay. Preferably, antibodies that specifically bind to hematopoietin receptor-like protein polypeptides do not detect other proteins in immunochemical assays and can immunoprecipitate a human hematopoietin receptor-like protein polypeptide from solution. See WO 01/98340.

Antisense Oligonucleotides

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of hematopoietin receptor-like protein gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester intemucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of hematopoietin receptor-like protein gene expression can be obtained by designing antisense oligonucleotides that will form duplexes to the control, 5′, or regulatory regions of the hematopoietin receptor-like protein gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes. See WO 01/98340.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a human hematopoietin receptor-like protein polynucleotide can be used to generate ribozymes that will specifically bind to mRNA transcribed from the hematopoietin receptor-like protein polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201). See WO 01/98340.

Differentially Expressed Genes

Described herein are methods for the identification of genes whose products interact with human hematopoietin receptor-like protein. Such genes may represent genes that are differentially expressed in disorders including, but not limited to, CNS disorders, COPD, cardiovascular disorders, liver disorders, cancer, asthma, and hematological disorders. Further, such genes may represent genes that are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, the human hematopoietin receptor-like protein gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

To identify differentially expressed genes total RNA or, preferably, mRNA is isolated from tissues of interest. For example, RNA samples are obtained from tissues of experimental subjects and from corresponding tissues of control subjects. Any RNA isolation technique that does not select against the isolation of mRNA may be utilized for the purification of such RNA samples. See, for example, Ausubel et al., ed., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples that represent RNA produced by differentially expressed genes are identified by methods well known to those of skill in the art. They include, for example, differential screening (Tedder et al., Proc. Natl. Acad. Sci. USA. 85, 208-12, 1988), subtractive hybridization (Hedrick et al., Nature 308, 149-53; Lee et al., Proc. Natl. Acad. Sci. USA. 88, 2825, 1984), and, preferably, differential display (Liang & Pardee, Science 257, 967-71, 1992; U.S. Pat. No. 5,262,311).

The differential expression information may itself suggest relevant methods for the treatment of disorders involving the human hematopoietin receptor-like protein. For example, treatment may include a modulation of expression of the differentially expressed genes and/or the gene encoding the human hematopoietin receptor-like protein. The differential expression information may indicate whether the expression or activity of the differentially expressed gene or gene product or the human hematopoietin receptor-like protein gene or gene product are up-regulated or down-regulated.

Screening Methods

The invention provides assays for screening test compounds that bind to or modulate the activity of a human hematopoietin receptor-like polypeptide or a human hematopoietin receptor-like polynucleotide. A test compound preferably binds to a human hematopoietin receptor-like polypeptide or polynucleotide. More preferably, a test compound decreases or increases functional activity by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. USA. 90, 6909, 1993; Erb et al. Proc. Natl. Acad. Sci. USA. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. USA. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to hematopoietin receptor-like polypeptides or polynucleotides or to affect hematopoietin receptor-like protein activity or hematopoietin receptor-like gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. US.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at. the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule that binds to the hematopoietin receptor-like polypeptide, such that normal biological activity is prevented. Examples of such small molecules include, but are not limited to, small peptides or peptide-like molecules.

In binding assays, either the test compound or the hematopoietin receptor-like polypeptide can comprise a detectable label, such as a fluorescent, radioisotopic, chemiluminescent, or enzymatic label, such as horseradish peroxidase, alkaline phosphatase, or luciferase. Detection of a test compound that is bound to the hematopoietin receptor-like polypeptide can then be accomplished, for example, by direct counting of radioemmission, by scintillation counting, or by determining conversion of an appropriate substrate to a detectable product.

Alternatively, binding of a test compound to a human hematopoietin receptor-like polypeptide can be determined without labeling either of the interactants. For example, a microphysiometer can be used to detect binding of a test compound with a human hematopoietin receptor-like polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a human hematopoietin receptor-like polypeptide (McConnell et al., Science 257, 1906-1912, 1992).

Determining the ability of a test compound to bind to a human hematopoietin receptor-like polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a human hematopoietin receptor-like polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent WO 94/10300), to identify other proteins which bind to or interact with the hematopoietin receptor-like polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a human hematopoietin receptor-like polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein that interacts with the hematopoietin receptor-like polypeptide.

It may be desirable to immobilize either the hematopoietin receptor-like polypeptide (or polynucleotide) or the test compound to facilitate separation of bound from unbound forms of one or both of the interactants, as well as to accommodate automation of the assay. Thus, either the hematopoietin receptor-like polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a human hematopoietin receptor-like polypeptide (or polynucleotide) can be accomplished in any vessel suitable for containing the reactants. Examples of such vessels include microtiter plates, test tubes, and microcentrifuge tubes.

In one embodiment, the hematopoietin receptor-like polypeptide is a fusion protein comprising a domain that allows the hematopoietin receptor-like polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed hematopoietin receptor-like polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a human hematopoietin receptor-like polypeptide (or polynucleotide) or a test compound can be immobilized utilizing conjugation of biotin and streptavidin. Biotinylated hematopoietin receptor-like polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates (Pierce Chemical). Alternatively, antibodies which specifically bind to a hematopoietin receptor-like polypeptide, polynucleotide, or a test compound, but which do not interfere with a desired binding site can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the hematopoietin receptor-like polypeptide or test compound, enzyme-linked assays which rely on detecting an activity of the hematopoietin receptor-like polypeptide, and SDS gel electrophoresis under nonreducing conditions.

Screening for test compounds which bind to a human hematopoietin receptor-like polypeptide or polynucleotide also can be carried out in an intact cell. Any cell which comprises a hematopoietin receptor-like polypeptide or polynucleotide can be used in a cell-based assay system. A hematopoietin receptor-like polynucleotide can be naturally occurring in the cell or can be introduced using techniques such as those described above. Binding of the test compound to a hematopoietin receptor-like polypeptide or polynucleotide is determined as described above.

Functional Activity

Test compounds can be tested for the ability to increase or decrease the functional activity of a human hematopoietin receptor-like polypeptide. Functional assays can be carried out after contacting either a purified hematopoietin receptor-like polypeptide, a cell membrane preparation, or an intact cell with a test compound. A test compound that decreases functional activity of a human hematopoietin receptor-like polypeptide by at least about 10, preferably about 50, more preferably about 75, 90, or 100% is identified as a potential therapeutic agent for decreasing hematopoietin receptor-like protein activity. A test compound which increases functional activity of a human hematopoietin receptor-like polypeptide by at least about 10, preferably about 50, more preferably about 75, 90, or 100% is identified as a potential therapeutic agent for increasing human hematopoietin receptor-like protein activity.

Gene Expression

In another embodiment, test compounds that increase or decrease hematopoietin receptor-like gene expression are identified. A hematopoietin receptor-like polynucleotide is contacted with a test compound, and the expression of an RNA or polypeptide product of the hematopoietin receptor-like polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of hematopoietin receptor-like mRNA or polypeptide expression in the cells can be determined by methods well known in the art for detecting mRNA or polypeptide. Either qualitative or quantitative methods can be used. The presence of polypeptide products of a human hematopoietin receptor-like polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a human hematopoietin receptor-like polypeptide.

Such screening can be carried out either in a cell-free assay system or in an intact cell. Any cell that expresses a human hematopoietin receptor-like polynucleotide can be used in a cell-based assay system. The hematopoietin receptor-like polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions that can be administered to a patient to achieve a therapeutic effect. Pharmaceutical compositions of the invention can comprise, for example, a human hematopoietin receptor-like polypeptide, hematopoietin receptor-like polynucleotide, ribozymes or antisense oligonucleotides, antibodies which specifically bind to a hematopoietin receptor-like polypeptide, or mimetics, activators, or inhibitors of a human hematopoietin receptor-like polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries that facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances that increase the viscosity of the suspension, such as sodium carboxyrnethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Human hematopoietin receptor-like protein can be regulated to treat CNS disorders, COPD, cardiovascular disorders, liver disorders, cancer, asthma, and hematological disorders.

Central Nervous System (CNS) Disorders

The novel human hematopoietin receptor-like protein is highly expressed in the following brain tissues: postcentral gyrus, dorsal root ganglia, retina, Alzheimer brain frontal lobe, cerebral meninges, neuroblastoma M32 cells, cerebellum (left), cerebellum (right), cerebral cortex, Alzheimer brain. The expression in brain tissues and in particular the differential expression between diseased tissue Alzheimer brain frontal lobe and healthy tissue frontal lobe, between diseased tissue Alzheimer brain and healthy tissue brain demonstrates that the novel human hematopoietin receptor-like protein or mRNA can be used to diagnose nervous system diseases. Additionally, the activity of the novel human hematopoietin receptor-like protein can be modulated to treat nervous system diseases.

Central and peripheral nervous system disorders also can be treated, such as primary and secondary disorders after brain injury, disorders of mood, anxiety disorders, disorders of thought and volition, disorders of sleep and wakefulness, diseases of the motor unit, such as neurogenic and myopathic disorders, neurodegenerative disorders such as Alzheimer's and Parkinson's disease, and processes of peripheral and chronic pain.

Pain that is associated with CNS disorders also can be treated by regulating the activity of human hematopoietin receptor-like protein. Pain which can be treated includes that associated with central nervous system disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation). Non-central neuropathic pain includes that associated with post mastectomy pain, reflex sympathetic dystrophy (RSD), trigeminal neuralgiaradioculopathy, post-surgical pain, HV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia. Pain associated with cancer and cancer treatment also can be treated, as can headache pain (for example, migraine with aura, migraine without aura, and other migraine disorders), episodic and chronic tension-type headache, tension-type like headache, cluster headache, and chronic paroxysmal hemicrania.

Cardiovascular Disorders

The novel human hematopoietin receptor-like protein is highly expressed in the following cardiovascular related tissues: aorta, heart ventricle (left), artery, HUVEC cells, vein, aorta sclerotic, heart atrium (left). Expression in the above mentioned tissues and in particular the differential expression between diseased tissue aorta sclerotic and healthy tissue aorta demonstrates that the novel human hematopoietin receptor-like protein or mRNA can be used to diagnose cardiovascular diseases. Additionally, the activity of the novel human hematopoietin receptor-like protein can be modulated to treat cardiovascular diseases.

Cardiovascular diseases include the following disorders of the heart and the vascular system: congestive heart failure, myocardial infarction, ischemic diseases of the heart, all kinds of atrial and ventricular arrhythmias, hypertensive vascular diseases, and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure, such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included, as well as the acute treatment of MI and the prevention of complications.

Ischemic diseases are conditions in which the coronary flow is restricted resulting in a perfusion which inadequate to meet the myocardial requirement for oxygen. This group of diseases includes stable angina, unstable angina, and asymptomatic ischemia.

Arrhythmias include all forms of atrial and ventricular tachyarrhythnias (atrial tachycardia, atrial flutter, atrial fibrillation, atrio-ventricular reentrant tachycardia, preexcitation syndrome, ventricular tachycardia, ventricular flutter, and ventricular fibrillation), as well as bradycardic forms of arrhythmias.

Vascular diseases include primary as well as all kinds of secondary arterial hypertension (renal, endocrine, neurogenic, others). The disclosed gene and its product may be used as drug targets for the treatment of hypertension as well as for the prevention of all complications. Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon, and venous disorders.

Liver Disorders

The novel human hematopoietin receptor-like protein is highly expressed in the following liver tissues: liver cirrhosis, liver tumor. The expression in liver tissues and in particular the differential expression between diseased tissue liver cirrhosis and healthy tissue liver demonstrates that the novel human hematopoietin receptor-like protein or mRNA can be used to diagnose liver diseases. Additionally, the activity of the novel human hematopoietin receptor-like protein can be modulated to treat those diseases.

Liver diseases comprise primary or secondary, acute or chronic diseases or injury of the liver which may be acquired or inherited, benign or malignant, and which may affect the liver or the body as a whole. They include but are not limited to disorders of the bilirubin metabolism, jaundice, syndromes of Gilbert's, Crigler-Najjar, Dubin-Johnson and Rotor, intrahepatic cholestasis, hepatomegaly, portal hypertension, ascites, Budd-Chiari syndrome, portal-systemic encephalopathy, fatty liver, steatosis, Reye's syndrome, liver diseases due to alcohol, alcoholic hepatitis or cirrhosis, fibrosis and cirrhosis, fibrosis and cirrhosis of the liver due to inborn errors of metabolism or exogenous substances, storage diseases, syndromes of Gaucher's, Zellweger's, Wilson's disease, acute or chronic hepatitis, viral hepatitis and its variants, inflammatory conditions of the liver due to viruses, bacteria, ftngi, protozoa, helminths; drug induced disorders of the liver, chronic liver diseases like primary sclerosing cholangitis, alphal-antitrypsin deficiency, primary biliary cirrhosis, postoperative liver disorders like postoperative intrahepatic cholestasis, hepatic granulomas, vascular liver disorders associated with systemic disease, benign or malignant neoplasms of the liver, disturbance of liver metabolism in the newborn or prematurely born.

Cancer

The novel human hematopoietin receptor-like protein is highly expressed in the following cancer tissues: lung tumor, breast tumor, stomach tumor, liver tumor, thyroid tumor, ileum tumor, esophagus tumor. The expression in the above mentioned tissues and in particular the differential expression between diseased tissue lung tumor and healthy tissue lung, between diseased tissue breast tumor and healthy tissue breast, between diseased tissue stomach tumor and healthy tissue stomach, between diseased tissue liver tumor and healthy tissue liver, between diseased tissue thyroid tumor and healthy tissue thyroid, between diseased tissue esophagus tumor and healthy tissue esophagus demonstrates that the novel human hematopoietin receptor-like protein or mRNA can be used to diagnose cancer. Additionally, the activity of the novel human hematopoietin receptor-like protein can be modulated to treat cancer.

Cancer disorders within the scope of the invention comprise any disease of an organ or tissue in mammals characterized by poorly controlled or uncontrolled multiplication of normal or abnormal cells in that tissue and its effect on the body as a whole. Cancer diseases within the scope of the invention comprise benign neoplasms, dysplasias, hyperplasias as well as neoplasms showing metastatic growth or any other transformations, e.g., leukoplakias, which often precede a breakout of cancer. Cells and tissues are cancerous when they grow more rapidly than normal cells, displacing or spreading into the surrounding healthy tissue or any other tissues of the body described as metastatic growth, assume abnormal shapes and sizes, show changes in their nucleocytoplasmatic ratio, nuclear polychromasia, and finally may cease.

Cancerous cells and tissues may affect the body as a whole when causing paraneoplastic syndromes or if cancer occurs within a vital organ or tissue, normal function will be impaired or halted, with possible fatal results. The ultimate involvement of a vital organ by cancer, either primary or metastatic, may lead to the death of the mammal affected. Cancer tends to spread, and the extent of its spread is usually related to an individual's chances of surviving the disease. Cancers are generally said to be in one of three stages of growth: early, or localized, when a tumor is still confined to the tissue of origin, or primary site; direct extension, where cancer cells from the tumor have invaded adjacent tissue or have spread only to regional lymph nodes; or metastasis, in which cancer cells have migrated to distant parts of the body from the primary site, via the blood or lymph systems, and have established secondary sites of infection. Cancer is said to be malignant because of its tendency to cause death if not treated.

Benign tumors usually do not cause death, although they may if they interfere with a normal body function by virtue of their location, size, or paraneoplastic side effects. Hence, benign tumors fall under the definition of cancer within the scope of the invention as well. In general, cancer cells divide at a higher rate than do normal cells, but the distinction between the growth of cancerous and normal tissues is not so much the rapidity of cell division in the former as it is the partial or complete loss of growth restraint in cancer cells and their failure to differentiate into a useful, limited tissue of the type that characterizes the functional equilibrium of growth of normal tissue.

Cancer tissues may express certain molecular receptors and probably are influenced by the host's susceptibility and immunity and it is known that certain cancers of the breast and prostate, for example, are considered dependent on specific hormones for their existence. The term “cancer” under the scope of the invention is not limited to simple benign neoplasia but includes any other benign and malign neoplasia, such as 1) carcinoma, 2) sarcoma, 3) carcinosarcoma, 4) cancers of the blood-forming tissues, 5) tumors of nerve tissues including the brain, and 6) cancer of skin cells.

Carcinoma occurs in epithelial tissues, which cover the outer body (the skin) and line mucous membranes and the inner cavitary structures of organs e.g. such as the breast, lung, the respiratory and gastrointestinal tracts, the endocrine glands, and the genitourinary system. Ductal or glandular elements may persist in epithelial tumors, as in adenocarcinomas, e.g., thyroid adenocarcinoma, gastric adenocarcinoma, uterine adenocarcinoma. Cancers of the pavement-cell epithelium of the skin and of certain mucous membranes, such as cancers of the tongue, lip, larynx, urinary bladder, uterine cervix, or penis, may be termed epidermoid or squamous-cell carcinomas of the respective tissues and are within the scope of the definition of cancer as well.

Sarcomas develop in connective tissues, including fibrous tissues, adipose (fat) tissues, muscle, blood vessels, bone, and cartilage such as osteogenic sarcoma, liposarcoma, fibrosarcoma, and synovial sarcoma.

Carcinosarcoma is cancer that develops in both epithelial and connective tissue. Cancer disease within the scope of this definition may be primary or secondary, whereby primary indicates that the cancer originated in the tissue where it is found rather than was established as a secondary site through metastasis from another lesion. Cancers and tumor diseases within the scope of this definition may be benign or malign and may affect all anatomical structures of the body of a mammal. By example, to they comprise cancers and tumor diseases of I) the bone marrow and bone marrow derived cells (leukemias), II) the endocrine and exocrine glands, such as the thyroid, parathyroid, pituitary, adrenal glands, salivary glands, and pancreas III) the breast, such as benign or malignant tumors in the mammary glands of either a male or a female, the mammary ducts, adenocarcinoma, medullary carcinoma, comedocarcinoma, Paget's disease of the nipple, inflammatory carcinoma of the young woman, IV) the lung, V) the stomach, VI) the liver and spleen, VII) the small intestine, VIII) the colon, IX) the bone and its supportive and connective tissues such as malignant or benign bone tumor, such as malignant osteogenic sarcoma, benign osteoma, cartilage tumors, malignant chondrosarcoma or benign chondroma,; bone marrow tumors such as malignant myeloma or benign eosinophilic granuloma, as well as metastatic tumors from bone tissues at other locations of the body; X) the mouth, throat, larynx, and the esophagus, XI) the urinary bladder and the internal and external organs and structures of the urogenital system of male and female such as the ovaries, uterus, cervix of the uterus, testes, and prostate gland, XII) the prostate, XIII) the pancreas, such as ductal carcinoma of the pancreas; XIV) the lymphatic tissue such as lymphomas and other tumors of lymphoid origin, XV) the skin, XVI) cancers and tumor diseases of all anatomical structures belonging to the respiratory systems including thoracal muscles and linings, XVII) primary or secondary cancer of the lymph nodes, XVIII) the tongue and of the bony structures of the hard palate or sinuses, XVIV) the mouth, cheeks, neck and salivary glands, XX) the blood vessels including the heart and their linings, XXI) the smooth or skeletal muscles and their ligaments and linings, XXII) the peripheral, the autonomous, the central nervous system including the cerebellum, and XXIII) the adipose tissue.

Hematological Disorders

The novel human hematopoietin receptor-like protein is highly expressed in the following tissues of the hematological system: thrombocytes, lymph node, bone marrow CD15⁺ cells, cord blood CD71⁺ cells. The expression in the above mentioned tissues demonstrates that the novel human hematopoietin receptor-like protein or mRNA can be used to diagnose hematological diseases. Additionally, the activity of the novel human hematopoietin receptor-like protein can be modulated to treat hematological disorders.

Anemia

Hemoglobin in red blood cells is the key component for transporting oxygen from the lungs to the tissues. In anemia the level of hemoglobin has fallen below 12 g/L. Therefore the oxygen carrying capacity of blood is reduced. Common reasons for anemia include acute or chronic blood loss, insufficient levels of erythropoietin synthesis in the kidneys (e.g. in dialysis patients) or insufficient output of red blood cells from bone marrow after chemotherapy or HIV infection etc. Current therapy of anemia is aimed at increasing the hematocrit either by transfusion or by stimulating erythropoiesis with agents such as erythropoietin. The treatment goal is to restore hemoglobin levels above 12 g/L.

Neutropenia

Neutrophils play a key role in the defense against infections. Neutropenia is an abnormally low white blood cell count which causes an increased incidence of infections. Causes of neutropenia include: drug-induced (e.g., following cancer chemotherapy), increased destruction of neutrophils (e.g., immune-mediated) or decreased bone marrow function (e.g., familial neutropenia). Neutropenia following cancer chemotherapy is currently treated with growth factors such as G-CSF or GM-CSF that stimulate granulopoiesis. The treatment goal is to raise the neutrophil count in order to reduce the susceptibility to infection.

Thrombocytopenia

Thrombocytopenia is a disorder where the number of platelets is inappropriately low. Since platelets play an essential role in thrombus formation to limit blood loss following vessel injury, insufficient platelet levels may lead to abnormal bleeding. There are many causes of thrombocytopenia including drug-induced thrombocytopenia (e.g., following cancer chemotherapy) and immune thromboytopenia (due to increased degradation of platelets). Platelet transfusions or IL-11 can be used to restore platelet levels in order to reduce the bleeding risk.

Aplastic Anemia (Pancyteponia)

Aplastic anemia is a life-threatening hematologic disorder characterized by absent or markedly diminished hematopoietic precursors in the bone marrow and resulting in neutropenia, anemia and thrombocytopenia. A large number of agents can cause aplastic anemia (drugs, chemicals and toxins) radiation and certain infections can also induce aplastic anemia. More frequently, aplastic anemia occurs as an unpredictable idiosyncratic reaction to drugs such as Antiinflammatory agents, antibiotics, and antiepileptic drugs. Aplastic anemia typically develops weeks or month during drug administration or delayed after drug administration has been discontinued. Several congenital and familiar forms of aplastic anemia have been described, including Fanconi's anemia, Shwachman-Diamond syndrome, familiar aplastic anemia, and aplasia associated with dyskeratosis congenita or amegakaryocytic thrompocytopenia.

COPD/and Asthma

The novel human hematopoietin receptor-like protein is highly expressed in the following tissues of the respiratory system: lung tumor, lung COPD, fetal lung. The expression in the above mentioned tissues and in particular the differential expression between diseased tissue lung COPD and healthy tissue lung demonstrates that the novel human hematopoietin receptor-like protein or mRNA can be used to diagnose COPD and/or asthma. Additionally, the activity of the novel human hematopoietin receptor-like protein can be modulated to treat those diseases.

Allergy is a complex process in which environmental antigens induce clinically adverse reactions. The inducing antigens, called allergens, typically elicit a specific IgE response and, although in most cases the allergens themselves have little or no intrinsic toxicity, they induce pathology when the IgE response in turn elicits an IgE-dependent or T cell-dependent hypersensitivity reaction. Hypersensitivity reactions can be local or systemic and typically occur within minutes of allergen exposure in individuals who have previously been sensitized to an allergen. The hypersensitivity reaction of allergy develops when the allergen is recognized by IgE antibodies bound to specific receptors on the surface of effector cells, such as mast cells, basophils, or eosinophils, which causes the activation of the effector cells and the release of mediators that produce the acute signs and symptoms of the reactions. Allergic diseases include asthma, allergic rhinitis (hay fever), atopic dermatitis, and anaphylaxis.

Asthma is though to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness caused by a decreased control of airway caliber, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to the tissue destruction related to the disorder. Other resident cells, such as smooth muscle cells, lung epithelial cells, mucus-producing cells, and nerve cells may also be abnormal in individuals with asthma and may contribute to the pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic disabling disorder requiring long-term management.

Despite recent important advances in our understanding of the pathophysiology of asthma, the disease appears to be increasing in prevalence and severity (Gergen and Weiss, Am. Rev. Respir. Dis. 146, 823-24, 1992). It is estimated that 30-40% of the population suffer with atopic allergy, and 15% of children and 5% of adults in the population suffer from asthma (Gergen and Weiss, 1992). Thus, an enormous burden is placed on our health care resources. However, both diagnosis and treatment of asthma are difficult. The severity of lung tissue inflammation is not easy to measure and the symptoms of the disease are often indistinguishable from those of respiratory infections, chronic respiratory inflammatory disorders, allergic rhinitis, or other respiratory disorders. Often, the inciting allergen cannot be determined, making removal of the causative environmental agent difficult.

Current pharmacological treatments suffer their own set of disadvantages. Commonly used therapeutic agents, such as beta agonists, can act as symptom relievers to transiently improve pulmonary function, but do not affect the underlying inflammation. Agents that can reduce the underlying inflammation, such as antiinflammatory steroids, can have major drawbacks that range from immunosuppression to bone loss (Goodman and Gilman's THE PHARMACOLOGIC BASIS OF THERAPEUTICS, Seventh Edition, MacMillan Publishing Company, NY, USA, 1985). In addition, many of the present therapies, such as inhaled corticosteroids, are short-lasting, inconvenient to use, and must be used often on a regular basis, in some cases for life, making failure of patients to comply with the treatment a major problem and thereby reducing their effectiveness as a treatment.

Because of the problems associated with conventional therapies, alternative treatment strategies have been evaluated. Glycophorin A (Chu and Sharom, Cell. Immunol. 145, 223-39, 1992), cyclosporin (Alexander et al., Lancet 339, 324-28, 1992), and a nonapeptide fragment of IL-2 (Zav'yalov et al., Immunol. Lett. 31, 285-88, 1992) all inhibit interleukin-2 dependent T lymphocyte proliferation; however, they are known to have many other effects. For example, cyclosporin is used as a immunosuppressant after organ transplantation. While these agents may represent alternatives to steroids in the treatment of asthmatics, they inhibit interleukin-2 dependent T lymphocyte proliferation and potentially critical immune functions associated with homeostasis.

Other treatments that block the release or activity of mediators of bronchochonstriction, such as cromones or anti-leukotrienes, have recently been introduced for the treatment of mild asthma, but they are expensive and not effective in all patients and it is unclear whether they have any effect on the chronic changes associated with asthmatic inflammation. What is needed in the art is the identification of a treatment that can act in pathways critical to the development of asthma that both blocks the episodic attacks of the disorder and preferentially dampens the hyperactive allergic immune response without inmmunocompromising the patient.

Chronic obstructive pulmonary (or airways) disease (COPD) is a condition defined physiologically as airflow obstruction that generally results from a mixture of emphysema and peripheral airway obstruction due to chronic bronchitis (Senior & Shapiro, Pulmonary Diseases and Disorders, 3d ed., New York, McGraw-Hill, 1998, pp. 659-681, 1998; Barnes, Chest 117, 10S-14S, 2000). Emphysema is characterized by destruction of alveolar walls leading to abnormal enlargement of the air spaces of the lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does occur in non-smokers.

Chronic inflammation of the airways is a key pathological feature of COPD (Senior & Shapiro, 1998). The inflammatory cell population comprises increased numbers of macrophages, neutrophils, and CD8⁺ lymphocytes. Inhaled irritants, such as cigarette smoke, activate macrophages which are resident in the respiratory tract, as well as epithelial cells leading to release of chemokines (e.g., interleukin-8) and other chemotactic factors. These chemotactic factors act to increase the neutrophil/monocyte trafficking from the blood into the lung tissue and airways. Neutrophils and monocytes recruited into the airways can release a variety of potentially damaging mediators such as proteolytic enzymes and reactive oxygen species. Matrix degradation and emphysema, along with airway wall thickening, surfactant dysfimction, and mucus hypersecretion, all are potential sequelae of this inflammatory response that lead to impaired airflow and gas exchange.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a human hematopoietin receptor-like polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects hematopoietin receptor-like protein activity can be administered to a human cell, either in vitro or in vivo, to reduce hematopoietin receptor-like protein activity. The reagent preferably binds to an expression product of a human hematopoietin receptor-like gene. If the expression product is a protein, the reagent is preferably an antibody. For treatment of human cells ex vivo, an antibody can be added to a preparation of stem cells that have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases functional activity relative to the functional activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors that can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides that express antisense oligonucleotides or ribozymes can be introduced into cells by a variety of methods, as described above.

Preferably, a reagent reduces expression of a human hematopoietin receptor-like gene or the activity of a hematopoietin receptor-like polypeptide by at least about 10, preferably about 50, more preferably about 75, 90, or 100% relative to the absence of the reagent. The effectiveness of the mechanism chosen to decrease the level of expression of a human hematopoietin receptor-like gene or the activity of a human hematopoietin receptor-like polypeptide can be assessed using methods well known in the art, such as hybridization of nucleotide probes to hematopoietin receptor-like protein-specific mRNA, quantitative RT-PCR, immunologic detection of a human hematopoietin receptor-like polypeptide, or measurement of functional activity.

In any of the embodiments described above any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human hematopoietin receptor-like protein also can be used in diagnostic assays for detecting diseases and abnormalities or susceptibility to diseases and abnormalities related to the presence of mutations in the nucleic acid sequences that encode the protein. For example, differences can be determined between the cDNA or genomic sequence encoding hematopoietin receptor-like protein in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of hematopoietin receptor-like protein also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLE 1

Detection of Hematopoietin Receptor-Like Protein Activity

The polynucleotide of SEQ ID NO: 3 or 4 is inserted into the expression vector pCEV4 and the expression vector pCEV4 hematopoietin receptor-like GPCR polypeptide obtained is transfected into human embryonic kidney 293 cells. The cells are scraped from a culture flask into 5 ml of Tris HCl, 5 mM EDTA, pH 7.5, and lysed by sonication. Cell lysates are centrifuged at 1000 rpm for 5 minutes at 4° C. The supernatant is centrifuged at 30,000×g for 20 minutes at 4° C. The pellet is suspended in binding buffer containing 50 mM Tris HCl, 5 mM MgSO₄, 1 mM EDTA, 100 mM NaCi, pH 7.5, supplemented with 0.1% BSA, 2 mg/ml aprotinin, 0.5 mg/ml leupeptin, and 10 mg/ml phosphoramidon. Optimal membrane suspension dilutions, defined as the protein concentration required to bind less than 10% of an added radioligand, i.e. hematopoietin are added to 96-well polypropylene microtiter plates containing ligand, non-labeled peptides, and binding buffer to a final volume of 250 ml.

In equilibrium saturation binding assays, membrane preparations are incubated in the presence of increasing concentrations (0.1 nM to 4 nM) of ¹²⁵I ligand.

Binding reaction mixtures are incubated for one hour at 30° C. The reaction is stopped by filtration through GF/B filters treated with 0.5% polyethyleneimine, using a cell harvester. Radioactivity is measured by scintillation counting, and data are analyzed by a computerized non-linear regression program. Non-specific binding is defined as the amount of radioactivity remaining after incubation of membrane protein in the presence of 100 nM of unlabeled peptide. Protein concentration is measured by the Bradford method using Bio-Rad Reagent, with bovine serum albumin as a standard. The hematopoietin receptor-like GPCR activity of the polypeptide comprising the amino acid sequence of SEQ ID NO: 2 (and 5 respectively) is demonstrated.

### EXAMPLE 2

Expression of Recombinant Human Hematopoietin Receptor-Like Protein

The Pichia pastoris expression vector pPICZB (Invitrogen, San Diego, Calif.) is used to produce large quantities of recombinant human hematopoietin receptor-like polypeptides in yeast. The hematopoietin receptor-like protein-encoding DNA sequence is derived from SEQ ID NO: 1 or 4. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, Calif.) according to manufacturer's instructions. Purified human hematopoietin receptor-like polypeptide is obtained.

### EXAMPLE 3

Identification of Test Compounds that Bind to Hematopoietin Receptor-Like Polypeptides

Purified hematopoietin receptor-like polypeptides comprising a glutathione-S-transferase protein and absorbed onto glutathione-derivatized wells of 96-well microtiter plates are contacted with test compounds from a small molecule library at pH 7.0 in a physiological buffer solution. Human hematopoietin receptor-like polypeptides comprise the amino acid sequence shown in SEQ ID NO: 2 or 5. The test compounds comprise a fluorescent tag. The samples are incubated for 5 minutes to one hour. Control samples are incubated in the absence of a test compound.

The buffer solution containing the test compounds is washed from the wells. Binding of a test compound to a human hematopoietin receptor-like polypeptide is detected by fluorescence measurements of the contents of the wells. A test compound that increases the fluorescence in a well by at least 15% relative to fluorescence of a well in which a test compound is not incubated is identified as a compound which binds to a human hematopoietin receptor-like polypeptide.

### EXAMPLE 4

Identification of a Test Compound Which Decreases Hematopoietin Receptor-Like Gene Expression

A test compound is administered to a culture of human cells transfected with a hematopoietin receptor-like protein expression construct and incubated at 37° C. for 10 to 45 minutes. A culture of the same type of cells that have not been transfected is incubated for the same time without the test compound to provide a negative control. RNA is isolated from the two cultures as described in Chirgwin et al., Biochem. 18, 5294-99, 1979). Northern blots are prepared using 20 to 30 μg total RNA and hybridized with a ³²P-labeled hematopoietin receptor-like protein-specific probe at 65° C. in Express-hyb (CLONTECH). The probe comprises at least 11 contiguous nucleotides selected from the complement of SEQ ID NO: 1 or 4. A test compound that decreases the hematopoietin receptor-like protein-specific signal relative to the signal obtained in the absence of the test compound is identified as an inhibitor of hematopoietin receptor-like gene expression.

### EXAMPLE 5

Identification of a Test Compound Which Decreases Hematopoietin Receptor-Like Protein Activity

A test compound is administered to a culture of human cells transfected with a hematopoietin receptor-like protein expression construct and incubated at 37° C. for 10 to 45 minutes. A culture of the same type of cells that have not been transfected is incubated for the same time without the test compound to provide a negative control.

A test compound which decreases the functional activity of the hematopoietin receptor-like protein relative to the functional activity in the absence of the test compound is identified as an inhibitor of hematopoietin receptor-like protein activity.

### EXAMPLE 6

Tissue-Specific Expression of Hematopoietin Receptor-Like Protein

The qualitative expression pattern of hematopoietin receptor-like protein in various tissues is determined by Reverse Transcription-Polymerase Chain Reaction (RT-PCR).

Quantitative Expression Profiling

To demonstrate that hematopoietin receptor-like protein is involved in CNS disorders, the following tissues are screened: fetal and adult brain, muscle, heart, lung, kidney, liver, thymus, testis, colon, placenta, trachea, pancreas, kidney, gastric mucosa, colon, liver, cerebellum, skin, cortex (Alzheimer's and normal), hypothalamus, cortex, amygdala, cerebellum, hippocampus, choroid, plexus, thalamus, and spinal cord.

To demonstrate that that hematopoietin receptor-like protein is involved in cancer, expression is determined in the following tissues: adrenal gland, bone marrow, brain, cerebellum, colon, fetal brain, fetal liver, heart, kidney, liver, lung, mammary gland, pancreas, placenta, prostate, salivary gland, skeletal muscle, small intestine, spinal cord, spleen, stomach, testis, thymus, thyroid, trachea, uterus, and peripheral blood lymphocytes. Expression in the following cancer cell lines also is determined: DU-145 (prostate), NCI-H125 (lung), HT-29 (colon), COLO-205 (colon), A-549 (lung), NCI-H460 (lung), HT-116 (colon), DLD-1 (colon), MDA-MD-231 (breast), LS174T (colon), ZF-75 (breast), MDA-MN-435 (breast), HT-1080, MCF-7 (breast), and U87. Matched pairs of malignant and normal tissue from the same patient also are tested.

To demonstrate that hematopoietin receptor-like protein is involved in the disease process of COPD, the initial expression panel consists of RNA samples from respiratory tissues and inflammatory cells relevant to COPD: lung (adult and fetal), trachea, freshly isolated alveolar type II cells, cultured human bronchial epithelial cells, cultured small airway epithelial cells, cultured bronchial sooth muscle cells, cultured H441 cells (Clara-like), freshly isolated neutrophils and monocytes, and cultured monocytes (macrophage-like). Body map profiling also is carried out, using total RNA panels purchased from Clontech. The tissues are adrenal gland, bone marrow, brain, colon, heart, kidney, liver, lung, mammary gland, pancreas, prostate, salivary gland, skeletal muscle, small intestine, spleen, stomach, testis, thymus, trachea, thyroid, and uterus.

To demonstrate that hematopoietin receptor-like protein is involved in the disease process of asthma, the following whole body panel is screened to show predominant or relatively high expression in lung or immune tissues: brain, heart, kidney, liver, lung, trachea, bone marrow, colon, small intestine, spleen, stomach, thymus, mammary gland, skeletal muscle, prostate, testis, uterus, cerebellum, fetal brain, fetal liver, spinal cord, placenta, adrenal gland, pancreas, salivary gland, thyroid, peripheral blood leukocytes, lymph node, and tonsil. Once this is established, the following lung and immune system cells are screened to localize expression to particular cell subsets: lung microvascular endothelial cells, bronchial/tracheal epithelial cells, bronchial/tracheal smooth muscle cells, lung fibroblasts, T cells (T helper 1 subset, T helper 2 subset, NKT cell subset, and cytotoxic T lymphocytes), B cells, mononuclear cells (monocytes and macrophages), mast cells, eosinophils, neutrophils, and dendritic cells. As a final step, the expression of hematopoietin receptor-like protein in cells derived from normal individuals with the expression of cells derived from asthmatic individuals is compared.

Quantitative expression profiling is performed by the form of quantitative PCR analysis called “kinetic analysis” firstly described in Higuchi et al., BioTechnology 10, 413-17, 1992, and Higuchi et al., BioTechnology 11, 1026-30, 1993. The principle is that at any given cycle within the exponential phase of PCR, the amount of product is proportional to the initial number of template copies.

If the amplification is performed in the presence of an internally quenched fluorescent oligonucleotide (TaqMan probe) complementary to the target sequence, the probe is cleaved by the 5′-3′ endonuclease activity of Taq DNA polymerase and a fluorescent dye released in the medium (Holland et al., Proc. Natl. Acad. Sci. U.S.A. 88, 7276-80, 1991). Because the fluorescence emission will increase in direct proportion to the amount of the specific amplified product, the exponential growth phase of PCR product can be detected and used to determine the initial template concentration (Heid et al., Genome Res. 6, 986-94, 1996, and Gibson et al., Genome Res. 6, 995-1001, 1996).

The amplification of an endogenous control can be performed to standardize the amount of sample RNA added to a reaction. In this kind of experiment, the control of choice is the 18S ribosomal RNA. Because reporter dyes with differing emission spectra are available, the target and the endogenous control can be independently quantified in the same tube if probes labeled with different dyes are used. All “real time PCR” measurements of fluorescence are made in the ABI Prism 7700.

RNA extraction and cDNA preparation. Total RNA from the tissues listed above are used for expression quantification. RNAs labeled “from autopsy” were extracted from autoptic tissues with the TRIzol reagent (Life Technologies, Md.) according to the manufacturer's protocol.

50 μg of each RNA were treated with DNase I for 1 hour at 37° C. in the following reaction mix: 0.2 U/μl RNase-free DNase I (Roche Diagnostics, Germany); 0.4 U/μl RNase inhibitor (PE Applied Biosystems, Calif.); 10 mM Tris-HCl pH 7.9; 10 mM MgCl₂; 50 mM NaCl; and 1 mM DTT.

After incubation, RNA is extracted once with 1 volume of phenol:chloroform:isoamyl alcohol (24:24:1) and once with chloroform, and precipitated with 1/10 volume of 3 M sodium acetate, pH 5.2, and 2 volumes of ethanol.

50 μg of each RNA from the autoptic tissues are DNase treated with the DNA-free kit purchased from Ambion (Ambion, Tex.). After resuspension and spectrophotometric quantification, each sample is reverse transcribed with the TaqMan Reverse Transcription Reagents (PE Applied Biosystems, Calif.) according to the manufacturer's protocol. The final concentration of RNA in the reaction mix is 200 ng/μL. Reverse transcription is carried out with 2.5 μM of random hexamer primers.

TaqMan quantitative analysis. Specific primers and probe are designed according to the recommendations of PE Applied Biosystems; the probe can be labeled at the 5′ end FAM (6-carboxy-fluorescein) and at the 3′ end with TAMR (6-carboxytetramethyl-rhodamine). Quantification experiments are performed on 10 ng of reverse transcribed RNA from each sample. Each determination is done in triplicate.

Total cDNA content is normalized with the simultaneous quantification (multiplex PCR) of the 18S ribosomal RNA using the Pre-Developed TaqMan Assay Reagents (PDAR) Control Kit (PE Applied Biosystems, Calif.).

The assay reaction mix is as follows: 1× final TaqMan Universal PCR Master Mix (from 2× stock) (PE Applied Biosystems, Calif.); 1× PDAR control−18S RNA (from 20× stock); 300 nM forward primer; 900 nM reverse primer; 200 nM probe; 10 ng cDNA; and water to 25 μl.

Each of the following steps are carried out once: pre PCR, 2 minutes at 50° C., and 10 minutes at 95° C. The following steps are carried out 40 times: denaturation, 15 seconds at 95° C., annealing/extension, 1 minute at 60° C.

The experiment is performed on an ABI Prism 7700 Sequence Detector (PE Applied Biosystems, Calif.). At the end of the run, fluorescence data acquired during PCR are processed as described in the ABI Prism 7700 user's manual in order to achieve better background subtraction as well as signal linearity with the starting target quantity.

### EXAMPLE 7

In Vivo Testing of Compounds/Target Validation

Pain

Acute pain. Acute pain is measured on a hot plate mainly in rats. Two variants of hot plate testing are used: In the classical variant animals are put on a hot surface (52 to 56° C.) and the latency time is measured until the animals show nocifensive behavior, such as stepping or foot licking. The other variant is an increasing temperature hot plate where the experimental animals are put on a surface of neutral temperature. Subsequently this surface is slowly but constantly heated until the animals begin to lick a hind paw. The temperature which is reached when hind paw licking begins is a measure for pain threshold.

Compounds are tested against a vehicle treated control group. Substance application is performed at different time points via different application routes (i.v., i.p., p.o., i.t., i.c.v., s.c., intradermal, transdermal) prior to pain testing.

Persistent pain. Persistent pain is measured with the formalin or capsaicin test, mainly in rats. A solution of 1 to 5% formalin or 10 to 100 μg capsaicin is injected into one hind paw of the experimental animal. After formalin or capsaicin application the animals show nocifensive reactions like flinching, licking and biting of the affected paw. The number of nocifensive reactions within a time frame of up to 90 minutes is a measure for intensity of pain.

Compounds are tested against a vehicle treated control group. Substance application is performed at different time points via different application routes (i.v., i.p., p.o., i.t., i.c.v., s.c., intradermal, transdermal) prior to formalin or capsaicin administration.

Neuropathic pain. Neuropathic pain is induced by different variants of unilateral sciatic nerve injury mainly in rats. The operation is performed under anesthesia. The first variant of sciatic nerve injury is produced by placing loosely constrictive ligatures around the common sciatic nerve. The second variant is the tight ligation of about the half of the diameter of the common sciatic nerve. In the next variant, a group of models is used in which tight ligations or transections are made of either the L5 and L6 spinal nerves, or the L % spinal nerve only. The fourth variant involves an axotomy of two of the three terminal branches of the sciatic nerve (tibial and common peroneal nerves) leaving the remaining sural nerve intact whereas the last variant comprises the axotomy of only the tibial branch leaving the sural and common nerves uninjured. Control animals are treated with a sham operation.

Postoperatively, the nerve injured animals develop a chronic mechanical allodynia, cold allodynioa, as well as a thermal hyperalgesia. Mechanical allodynia is measured by means of a pressure transducer (electronic von Frey Anesthesiometer, IITC Inc.-Life Science Instruments, Woodland Hills, SA, USA; Electronic von Frey System, Somedic Sales AB, Hörby, Sweden). Thermal hyperalgesia is measured by means of a radiant heat source (Plantar Test, Ugo Basile, Comerio, Italy), or by means of a cold plate of 5 to 10° C. where the nocifensive reactions of the affected hind paw are counted as a measure of pain intensity. A further test for cold induced pain is the counting of nocifensive reactions, or duration of nocifensive responses after plantar administration of acetone to the affected hind limb. Chronic pain in general is assessed by registering the circadanian rhythms in activity (Surjo and Arndt, Universität zu Köln, Cologne, Germany), and by scoring differences in gait (foot print patterns; FOOTPRINTS program, Klapdor et al., 1997. A low cost method to analyze footprint patterns. J. Neurosci. Methods 75, 49-54).

Compounds are tested against sham operated and vehicle treated control groups. Substance application is performed at different time points via different application routes (i.v., i.p., p.o., i.t., i.c.v., s.c., intradermal, transdermal) prior to pain testing.

Inflammatory Pain. Inflammatory pain is induced mainly in rats by injection of 0.75 mg carrageenan or complete Freund's adjuvant into one hind paw. The animals develop an edema with mechanical allodynia as well as thermal hyperalgesia. Mechanical allodynia is measured by means of a pressure transducer (electronic von Frey Anesthesiometer, IITC Inc.-Life Science Instruments, Woodland Hills, SA, USA). Thermal hyperalgesia is measured by means of a radiant heat source (Plantar Test, Ugo Basile, Comerio, Italy, Paw thermal stimulator, G. Ozaki, University of California, USA). For edema measurement two methods are being used. In the first method, the animals are sacrificed and the affected hindpaws sectioned and weighed. The second method comprises differences in paw volume by measuring water displacement in a plethysmometer (Ugo Basile, Comerio, Italy).

Compounds are tested against uninflamed as well as vehicle treated control groups. Substance application is performed at different time points via different application routes (i.v., i.p., p.o., i.t., i.c.v., s.c., intradermal, transdermal) prior. to pain testing.

Diabetic neuropathic pain. Rats treated with a single intraperitoneal injection of 50 to 80 mg/kg streptozotocin develop a profound hyperglycemia and mechanical allodynia within 1 to 3 weeks. Mechanical allodynia is measured by means of a pressure transducer (electronic von Frey Anesthesiometer, IITC Inc.-Life Science Instruments, Woodland Hills, SA, USA).

Compounds are tested against diabetic and non-diabetic vehicle treated control groups. Substance application is performed at different time points via different application routes (i.v., i.p., p.o., i.t., i.c.v., s.c., intradermal, transdermal) prior to pain testing.

Parkinson's Disease

6-Hydroxydopamine (6-OH-DA) Lesion. Degeneration of the dopaminergic nigrostriatal and striatopallidal pathways is the central pathological event in Parkinson's disease. This disorder has been mimicked experimentally in rats using single/sequential unilateral stereotaxic injections of 6-OH-DA into the medium forebrain bundle (MFB).

Male Wistar rats (Harlan Winkelmann, Germany), weighing 200±250 g at the beginning of the experiment, are used. The rats are maintained in a temperature- and humidity-controlled environment under a 12 h light/dark cycle with free access to food and water when not in experimental sessions. The following in vivo protocols are approved by the governmental authorities. All efforts are made to minimize animal suffering, to reduce the number of animals used, and to utilize alternatives to in vivo techniques.

Animals are administered pargyline on the day of surgery (Sigma, St. Louis, Mo., USA; 50 mg/kg. i.p.) in order to inhibit metabolism of 6-OHDA by monoamine oxidase and desmethylimipramine HCl (Sigma; 25 mg/kg i.p.) in order to prevent. uptake of 6-OHDA by noradrenergic terminals. Thirty minutes later the rats are anesthetized with sodium pentobarbital (50 mg/kg) and placed in a stereotaxic frame. In order to lesion the DA nigrostriatal pathway 4 μl of 0.01% ascorbic acid-saline containing 8 μg of 6-OHDA HBr (Sigma) are injected into the left medial fore-brain bundle at a rate of 1 μl/min (2.4 mm anterior, 1.49 mm lateral, −2.7 mm ventral to Bregma and the skull surface). The needle is left in place an additional 5 min to allow diffusion to occur.

Stepping Test. Forelimb akinesia is assessed three weeks following lesion placement using a modified stepping test protocol. In brief, the animals are held by the experimenter with one hand fixing the hindlimbs and slightly raising the hind part above the surface. One paw is touching the table, and is then moved slowly sideways (5 s for 1 m), first in the forehand and then in the backhand direction. The number of adjusting steps is counted for both paws in the backhand and forehand direction of movement. The sequence of testing is right paw forehand and backhand adjusting stepping, followed by left paw forehand and backhand directions. The test is repeated three times on three consecutive days, after an initial training period of three days prior to the first testing. Forehand adjusted stepping reveals no consistent differences between lesioned and healthy control animals. Analysis is therefore restricted to backhand adjusted stepping.

Balance Test. Balance adjustments following postural challenge are also measured during the stepping test sessions. The rats are held in the same position as described in the stepping test and, instead of being moved sideways, tilted by the experimenter towards the side of the paw touching the table. This maneuver results in loss of balance and the ability of the rats to regain balance by forelimb movements is scored on a scale ranging from 0 to 3. Score 0 is given for a normal forelimb placement. When the forelimb movement is delayed but recovery of postural balance detected, score 1 is given. Score 2 represents a clear, yet insufficient, forelimb reaction, as evidenced by muscle contraction, but lack of success in recovering balance, and score 3 is given for no reaction of movement. The test is repeated three times a day on each side for three consecutive days after an initial training period of three days prior to the first testing.

Staircase Test (Paw Reaching). A modified version of the staircase test is used for evaluation of paw reaching behavior three weeks following primary and secondary lesion placement. Plexiglass test boxes with a central platform and a removable staircase on each side are used. The apparatus is designed such that only the paw on the same side at each staircase can be used, thus providing a measure of independent forelimb use. For each test the animals are left in the test boxes for 15 min. The double staircase is filled with 7×3 chow pellets (Precision food pellets, formula: P, purified rodent diet, size 45 mg; Sandown Scientific) on each side. After each test the number of pellets eaten (successfully retrieved pellets) and the number of pellets taken (touched but dropped) for each paw and the success rate (pellets eaten/pellets taken) are counted separately. After three days of food deprivation (12 g per animal per day) the animals are tested for 11 days. Full analysis is conducted only for the last five days. MPTP treatment. The neurotoxin 1-methyl-4-phenyl-1,2,3,6-tetrahydropyridine (MPTP) causes degeneration of mesencephalic dopaminergic (DAergic) neurons in rodents, non-human primates, and humans and, in so doing, reproduces many of the symptoms of Parkinson's disease. MPTP leads to a marked decrease in the levels of dopamine and its metabolites, and in the number of dopaminergic terminals in the striatum as well as severe loss of the tyrosine hydroxylase (TH)-immunoreactive cell bodies in the substantia nigra, pars compacta.

In order to obtain severe and long-lasting lesions, and to reduce mortality, animals receive single injections of MPTP, and are then tested for severity of lesion 7-10 days later. Successive MPTP injections are administered on days 1, 2 and 3. Animals receive application of 4 mg/kg MPTP hydrochloride (Sigma) in saline once daily. All injections are intraperitoneal (i.p.) and the MPTP stock solution is frozen between injections. Animals are decapitated on day 11.

Immunohistology. At the completion of behavioral experiments, all animals are anaesthetized with 3 ml thiopental (1 g/40 ml i.p., Tyrol Pharma). The mice are perfused transcardially with 0.01 M PBS (pH 7.4) for 2 min, followed by 4% paraformaldehyde (Merck) in PBS for 15 min. The brains are removed and placed in 4% paraformaldehyde for 24 h at 4° C. For dehydration they are then transferred to a 20% sucrose (Merck) solution in 0.1 M PBS at 4° C. until they sink. The brains are frozen in methylbutan at −20° C. for 2 min and stored at −70° C. Using a sledge microtome (mod. 3800-Frigocut, Leica), 25 μm sections are taken from the genu of the corpus callosum (AP 1.7 mm) to the hippocampus (AP 21.8 mm) and from AP 24.16 to AP 26.72. Forty-six sections are cut and stored in assorters in 0.25 M Tris buffer (pH 7.4) for immunohistochemistry.

A series of sections is processed for free-floating tyrosine hydroxylase (TH) immunohistochemistry. Following three rinses in 0.1 M PBS, endogenous peroxidase activity is quenched for 10 min in 0.3% H₂O₂±PBS. After rinsing in PBS, sections are preincubated in 10% normal bovine serum (Sigma) for 5 min as blocking agent and transferred to either primary anti-rat TH rabbit antiserum (dilution 1:2000).

Following overnight incubation at room temperature, sections for TH immunoreactivity are rinsed in PBS (2×10 min) and incubated in biotinylated anti-rabbit immunoglobulin G raised in goat (dilution 1:200) (Vector) for 90 min, rinsed repeatedly and transferred to Vectastain ABC (Vector) solution for 1 h. 3,.3′-Diaminobenzidine tetrahydrochloride (DAB; Sigma) in 0.1 M PBS, supplemented with 0.005% H₂O₂, serves as chromogen in the subsequent visualization reaction. Sections are mounted on to gelatin-coated slides, left to dry overnight, counter-stained with hematoxylin dehydrated in ascending alcohol concentrations and cleared in butylacetate. Coverslips are mounted on entellan.

Rotarod Test. We use a modification of the procedure described by Rozas and Labandeira-Garcia (1997), with a CR-1 Rotamex system (Columbus Instruments, Columbus, Ohio) comprising an IBM-compatible personal computer, a CIO-24 data acquisition card, a control unit, and a four-lane rotarod unit. The rotarod unit consists of a rotating spindle (diameter 7.3 cm) and individual compartments for each mouse. The system software allows preprogramming of session protocols with varying rotational speeds (0-80 rpm). Infrared beams are used to detect when a mouse has fallen onto the base grid beneath the rotarod. The system logs the fall as the end of the experiment for that mouse, and the total time on the rotarod, as well as the time of the fall and all the set-up parameters, are recorded. The system also allows a weak current to be passed through the base grid, to aid training.

Dementia

The object recognition task. The object recognition task has been designed to assess the effects of experimental manipulations on the cognitive performance of rodents. A rat is placed in an open field, in which two identical objects are present. The rats inspects both objects during the first trial of the object recognition task. In a second trial, after a retention interval of for example 24 hours, one of the two objects used in the first trial, the ‘familiar’ object, and a novel object are placed in the open field. The inspection time at each of the objects is registered. The basic measures in the OR task is the time spent by a rat exploring the two object the second trial. Good retention is reflected by higher exploration times towards the novel than the ‘familiar’ object.

Administration of the putative cognition enhancer prior to the first trial predominantly allows assessment of the effects on acquisition, and eventually on consolidation processes. Administration of the testing compound after the first trial allows to assess the effects on consolidation processes, whereas administration before the second trial allows to measure effects on retrieval processes.

The passive avoidance task. The passive avoidance task assesses memory performance in rats and mice. The inhibitory avoidance apparatus consists of a two-compartment box with a light compartment and a dark compartment. The two compartments are separated by a guillotine door that can be operated by the experimenter. A threshold of 2 cm separates the two compartments when the guillotine door is raised. When the door is open, the illumination in the dark compartment is about 2 lux. The light intensity is about 500 lux at the center of the floor of the light compartment.

Two habituation sessions, one shock session, and a retention session are given, separated by inter-session intervals of 24 hours. In the habituation sessions and the retention session the rat is allowed to explore the apparatus for 300 sec. The rat is placed in the light compartment, facing the wall opposite to the guillotine door. After an accommodation period of 15 sec. the guillotine door is opened so that all parts of the apparatus can be visited freely. Rats normally avoid brightly lit areas and will enter the dark compartment within a few seconds.

In the shock session the guillotine door between the compartments is lowered as soon as the rat has entered the dark compartment with its four paws, and a scrambled 1 mA footshock is administered for 2 sec. The rat is removed from the apparatus and put back into its home cage. The procedure during the retention session is identical to that of the habituation sessions.

The step-through latency, that is the first latency of entering the dark compartment (in sec.) during the retention session is an index of the memory performance of the animal; the longer the latency to enter the dark compartment, the better the retention is. A testing compound in given half an hour before the shock session, together with 1 mg*kg⁻¹ scopolamine. Scopolamine impairs the memory performance during the retention session 24 hours later. If the test compound increases the enter latency compared with the scopolamine-treated controls, is likely to possess cognition enhancing potential.

The Morris water escape task. The Morris water escape task measures spatial orientation learning in rodents. It is a test system that has extensively been used to investigate the effects of putative therapeutic on the cognitive functions of rats and mice. The performance of an animal is assessed in a circular water tank with an escape platform that is submerged about 1 cm below the surface of the water. The escape platform is not visible for an animal swimming in the water tank. Abundant extra-maze cues are provided by the furniture in the room, including desks, computer equipment, a second water tank, the presence of the experimenter, and by a radio on a shelf that is playing softly.

The animals receive four trials during five daily acquisition sessions. A trial is started by placing an animal into the pool, facing the wall of the tank. Each of four starting positions in the quadrants north, east, south, and west is used once in a series of four trials; their order is randomized. The escape platform is always in the same position. A trial is terminated as soon as the animal had climbs onto the escape platform or when 90 seconds have elapsed, whichever event occurs first. The animal is allowed to stay on the platform for 30 seconds. Then it is taken from the platform and the next trial is started. If an animal did not find the platform within 90 seconds it is put on the platform by the experimenter and is allowed to stay there for 30 seconds. After the fourth trial of the fifth daily session, an additional trial is given as a probe trial: the platform is removed, and the time the animal spends in the four quadrants is measured for 30 or 60 seconds. In the probe trial, all animals start from the same start position, opposite to the quadrant where the escape platform had been positioned during acquisition.

Four different measures are taken to evaluate the performance of an animal during acquisition training: escape latency, traveled distance, distance to platform, and swimming speed. The following measures are evaluated for the probe trial: time (s) in quadrants and traveled distance (cm) in the four quadrants. The probe trial provides additional information about how well an animal learned the position of the escape platform. If an animal spends more time and swims a longer distance in the quadrant where the platform had been positioned during the acquisition sessions than in any other quadrant, one concludes that the platform position has been learned well.

In order to assess the effects of putative cognition enhancing compounds, rats or mice with specific brain lesions which impair cognitive functions, or animals treated with compounds such as scopolamine or MK-801, which interfere with normal learning, or aged animals which suffer from cognitive deficits, are used.

The T-maze spontaneous alternation task. The T-maze spontaneous alternation task (TeMCAT) assesses the spatial memory performance in mice. The start arm and the two goal arms of the T-maze are provided with guillotine doors which can be operated manually by the experimenter. A mouse is put into the start arm at the beginning of training. The guillotine door is closed. In the first trial, the ‘forced trial’, either the left or right goal arm is blocked by lowering the guillotine door. After the mouse has been released from the start arm, it will negotiate the maze, eventually enter the open goal arm, and return to the start position, where it will be confined for 5 seconds, by lowering the guillotine door. Then, the animal can choose freely between the left and right goal arm (all guillotine-doors opened) during 14 ‘free choice’ trials. As soon a the mouse has entered one goal arm, the other one is closed. The mouse eventually returns to the start arm and is free to visit whichever go alarm it wants after having been confined to the start arm for 5 seconds. After completion of 14 free choice trials in one session, the animal is removed from the maze. During training, the animal is never handled.

The percent alternations out of 14 trials is calculated. This percentage and the total time needed to complete the first forced trial and the subsequent 14 free choice trials (in s) is analyzed. Cognitive deficits are usually induced by an injection of scopolamine, 30 min before the start of the training session. Scopolamine reduced the per-cent alternations to chance level, or below. A cognition enhancer, which is always administered before the training session, will at least partially, antagonize the scopolamine-induced reduction in the spontaneous alternation rate.

### EXAMPLE 8

Identification of Test Compound Efficacy in an Animal Model of COPD

A/J mice are exposed to the smoke from 2 unfiltered cigarettes per day for 6 days per week for 14 weeks. Non-smoking, age-matched animals are used as controls. Animals are orally dosed with test compound or vehicle 1 hour before and 7 hours after smoke exposure. This twice-daily dosing regime is continued throughout the smoke exposure period. On day 7 of the weekly exposure, animals are given only 1 dose of test compound and are not exposed to cigarette smoke.

After the smoke exposure period, the mice are killed, their lungs inflated with phosphate-buffered formalin via their trachea, and then the lungs and heart are removed en bloc and fixed at 4° C. for 48 hours. The lungs are then prepared for paraffin wax sectioning, and 4 mm sections are cut and mounted on glass slides. Sections are then stained with haematoxylin and eosin. Morphometric analysis of lung sections is done by calculation of the Linear Mean Intercept (LMI) parameter using a semi-automated computer image analysis system. Each slide (1 per mouse) contains several sections originating from multiple lobes. Twelve non-overlapping areas (each area covering 1.53×10−3 cm²) are randomly selected for LMI analysis. The 12 areas cover a minimum of two lobes per slide. Non-parenchymal components (airways, blood vessels) are excluded from the analysis to prevent artifactual error. The mean intercept length is calculated for each mouse. Development of emphysema is seen as an increase in LMI.

LMI data are expressed as the median and statistical comparisons are done using the non-parametric Mann-Witney U-test. A ‘p’ value of <=0.05 is considered to be statistically significant. The potency of a test compound is evaluated by comparison of the tobacco smoke induced increase in LMI in animals dosed with either the test compound or just the vehicle used for administration of the compound.

### EXAMPLE 9

Identification of Test Compound Efficacy in an In Vitro Functional Test Relevant to COPD

The potency of test compounds is evaluated by measuring the inhibition of elastolysis induced by human alveolar macrophages. The cells are isolated from bronchoalveolar lavage samples taken from non-smokers, disease-free smokers, and smokers with COPD. Macrophage suspensions are added to test wells coated with tritiated elastin and incubated at 37° C. for 3 h to allow adherence of the cells. The wells are then carefully washed to remove non-adherent cells and fresh medium is added to each well. The cells are incubated at 37° C. for up to 72 hours in the presence or absence of test compound. Every 24 hours the medium in each well is removed for analysis and replaced by fresh medium. Radioactivity released into the medium is measured by liquid scintillation counting and the rate of elastin degradation is calculated. The potency of a test compound is evaluated by comparing the rate of elastolysis measured with cells incubated in the presence or absence of the compound.

### EXAMPLE 10

Atherosclerosis: In Vivo Target Validation

Effects on plasma cholesterol levels including HDL cholesterol are typically assessed in humanized apo-AI transgenic mice. Modulation of human target proteins can be determined in corresponding transgenic mice (e.g., CETP transgenic mice). Triglyceride lowering is usually evaluated in ob/ob mice or Zucker rats. Animals are fed with normal diets or modified diets (e.g., enriched by 0.5% cholesterol 20% coconut oil). Standard protocols consist of oral applications once daily for 7 to 10 days at doses ranging from 0,1 to 100 mg/kg. The compounds are dissolved (e.g., in Solutol/Ethanol/saline mixtures) and applied by oral gavage or intravenous injection. Before and at the end of the application period, blood samples are typically drawn by retroorbital punctuation. Plasma cholesterol and triglyceride levels are determined with standardized clinical diagnostic kits (e.g., INFINITY™ cholesterol reagent and INFINITY™ triglyceride reagent; Sigma, St. Louis). HDL cholesterol is determined after phosphotungstic acid precipitation of non-HDL lipoproteins or FPLC gel filtration with post-column derivatization of cholesterol using the reagents mentioned above. Plasma levels of human apolipoprotein-AI in relevant humanized transgenic mice are measured by immunoturbidimetry (Sigma).

Long-term anti-atherosclerotic potency of drug candidates are evaluated in Apo E-knockout mice. Therefore, animals are fed a standard chow diet (4,5% fat) or a Western diet (20% fat) containing 1 to 100 mg/kg of the respective compounds for 3 to 5 month. Arterial lesions are quantified in serial cryosections of the proximal aorta by staining with Oil Red O and counterstaining with hematoxylin. Lesion area size is determined using an digital imaging system.

### EXAMPLE 11

In Vivo Testing of Cardiovascular Effects of Test Compounds

Hemodynamics in Anesthetized Rats

Male Wistar rats weighing 300-350 g (Harlan Winkelmann, Borchen, Germany) are anesthetized with thiopental “Nycomed” (Nycomed, Munich, Germany) 100 mg kg⁻¹ i.p. A tracheotomy is performed, and catheters are inserted into the femoral artery for blood pressure and heart rate measurements (Gould pressure transducer and recorder, model RS 3400) and into the femoral vein for substance administration. The animals are ventilated with room air and their body temperature is controlled. Test compounds are administered orally or intravenously.

Hemodynamics in Conscious SHR

Female conscious SHR (Moellegaard/Denmark, 220-290 g) are equipped with implantable radiotelemetry, and a data aquisition system (Data Sciences, St. Paul, Minn., USA), comprising a chronically implantable transducer/transmitter unit equipped with a fluid-filled catheter is used. The transmitter is implanted into the peritoneal cavity, and the sensing catheter is inserted into the descending aorta.

Single administration of test compounds is performed as a solution in Transcutol®/Cremophor®/H₂O (10/20/70=v/v/v) given orally by gavage. The animals of control groups only receive the vehicle. Before treatment, mean blood pressure and heart rate of treated and untreated control groups are measured.

Hemodynamics in Anesthetized Dogs

Studies are performed on anesthetized dogs of either sex (body weight between 20-30 kg). Anesthesia is initiated by slow intravenous injection of 25 mg kg⁻¹ sodium thiopental (Trapanal®, Byk Gulden, Konstanz, Germany). The anesthesia is continued and maintained throughout the experiment by continuous infusion of 0.04 mg kg⁻¹h⁻¹ fentanyl (Fentanyl®, Janssen, Neuss, Germany) and 0.25 mg kg⁻¹h⁻¹, droperidol (DihydrobenzperidolR, Janssen, Neuss, Germany). During this anaesthesia, heart rate is as low as 35-40 bpm due to increased vagal tone. Therefore, a parasympathetic blockade is achieved by intermittent injections of atropine (0.1 mg per animal) (AtropinsulfatR, Eifelfango, Bad Neuenahr, Germany). After intubation the animals are artificially ventilated at constant volume (EngströmR 300, Engström, Sweden) with room air enriched with 30% oxygen to maintain an end-tidal CO₂ concentration of about 5% (NormocapR, Datex, Finland).

The following catheters are implanted for measurement of cardiovascular parameters: a tip catheter for recording of left ventricular pressure is inserted into the ventricle via the carotid artery (PC350, Millar Instruments, Houston, Tex., USA), a hollow catheter is inserted into the femoral artery and connected to a strain gauge (type 4-327-1, Telos Medical, Upland, Calif., USA for recording of arterial blood pressure, two venous catheters are inserted into either femoral vein and one additional catheter into a forearm vein for application of the anaesthetic and drugs, respectively, and an oxymetry catheter for recording of oxygen saturation is inserted into the coronary sinus via the jugular vein (Schwarzer IVH4, München, Germany).

After a left-sided thoracotomy the ramus circumflexus of the left coronary artery (LCX) is freed from connective tissue, and an electromagnetic flow probe (Gould Statham, Oxnard, Calif,, USA) is applied for measurement of coronary blood flow. Arterial blood pressure, electrocardiogram (lead II), left ventricular pressure, first derivative of left ventricular pressure (dP/dt), heart rate, coronary blood flow, and oxygen saturation in the coronary sinus are continuously recorded on a pen recorder (Brush, Gould, Cleveland, Ohio, USA). The maximum of dP/dt is used as measure of left ventricular contractility (dP/dtmax). After completion of the instrumentation, an interval of 60 min is allowed for stabilization before the test compound is intravenously applied as bolus injections. Care is taken that all measured cardiovascular parameters have returned to control level before injection of the next dose. Each dose of the test compound is tested at least three times in different animals. The order of injection of the different doses is randomized in each animal.

### EXAMPLE 12

Hematology: In Vitro Testing of Compounds/Target Validation

Isolation of CD34⁺ Cells

Mononuclear cells from fresh blood (cord blood, peripheral blood, bone marrow) were separated by Ficoll Paque® (1.077 density, Amersham-Pharmacia) density gradient centrifugation, and CD34⁺ cells were purified by immunomagnetic separation system (MiniMACS, Miltenyi Biotec), according to the manufacture's instructions (Direct CD34 Progenitor Cell Isolation Kit, Miltenyi Biotec). The percentage of CD34⁺ cells were generally from 90-95%.

Erythropoiesis/Anemia

Erythroid CD34⁺ Liquid Culture

1-2×10⁴ CD34⁺ cells were plated in triplicate in 24-well plates with 1 ml Iscoves modified Dulbecco medium (IMDM) (Invitrogen) containing 10% fetal bovine serum (FCS, Invitrogen), 1% Glutamine (Invitrogen) supplemented with SCF (25 ng/ml) (PeproTech), different concentration of Erythropoietin (0.01 U/ml-1 U/ml) (Erypo® FS 4000, Cilag) with or without compounds. Control cells were incubated with 0.1-0.2% DMSO instead of compounds. The cultures were incubated at 37° C. in a fully humidified atmosphere with 5% CO₂. After 9 to 14 days cells were harvested, counted and stained with phycoerythrin (PE)-conjugated mAb against Glycophorin A (Pharmingen) to analyze differentiation.

Erythroid Colony-Forming Assay

Five hundred CD34⁺ cells/ml were plated in triplicate 24-well plates with 1% methylcellulose in IMDM containing 30% FCS, 1% bovine serun albumin (BSA), 2 mM L-glutamine and 10-4 M 2-mercaptoethanol (Methocult H4230, Cell Systems®), IL-3 (10 ng/ml ) (PeproTech) with different concentration of erythro-poietin (0.01 U/ml-1 U/ml) with or without compounds. The cultures were incubated at 37° C. in a fully humidified atmosphere with 5% CO₂. After 9 to 14 days erythroid burst forrning units (BFU-E) were counted from each of the plates. Afterwards cells were dissolved from methylcellulose with 0.1% NaCl solution. Cells were counted and stained with phycoerythrin (PE)-conjugated mAb against Glycophorin A (Pharmingen) to analyze differentiation.

BFU-E Culture

1×10⁵ Cord Blood CD34⁺ cells/ml were cultured in IMDM containing 15% BIT-9500 (Cell Systems®), supplemented with IL-3 (10 ng/ml), IL-6 (10 ng/ml) and SCF (25 ng/ml) (PeproTech) and incubated at 37° C. in a fully humidified atmosphere with 5% CO₂. 3 and 5 days after initiation of culture an equal volume of fresh medium supplemented with 2× cytokines were added. On day 6 to 7 1-2×10⁴ erythroid progenitors were plated in triplicate in 24-well plates with 1 ml IMDM containing 10% FCS, 1% glutamine supplemented with SCF (25 ng/ml), different concentration of erythropoietin (0.01 U/ml-1 U/ml) with or without compounds. Control cells were incubated with 0.1-0.2% DMSO instead of compounds. The cultures were incubated at 37° C. in a fully humidified atmosphere with 5% CO₂. After 6 to 8 days cells were harvested and counted to analyze proliferation.

CD36⁺ Cells

1×10⁵ Cord Blood CD34⁺ cells/ml were cultured in IMDM containing 15% BIT-9500 supplemented with IL-3 (10 ng/ml), IL-6 (10 ng/ml) and SCF (25 ng/ml) and incubated at 37° C. in a fully humidified atmosphere with 5% CO₂. 3 and 5 days after initiation of culture an equal volume of fresh medium supplemented. with 2× cytokines were added. On day 6 to 7 cells were stained with PE-conjugated mAb against CD36 (Pharmingen) and CD36⁺ cells were purified using anti-PE microbeads and Mini MACS system (Miltenyi Biotec) according to the manufacture's instructions. 1-2×10⁴ CD36⁺ cells were plated in triplicate 24 well plates with 1 ml IMDM containing 10% FCS, 1% Glutaminie supplemented with SCF (25 ng/ml), different concentration of Erythropoietin (0.01 U/ml-1 U/ml) with or without compounds. Control cells were incubated with 0.1-0.2% DMSO instead of compounds. The cultures were incubated at 37° C. in a fully humidified atmosphere with 5% CO₂. After 6 to 8 days cells were harvested and counted to analyze proliferation.

Myelopoiesis and Thrombocytopoiesis

Myeloid CD34⁺ Liquid Culture

5×10³ CD34⁺ cells isolated from peripheral blood, cord blood or from bone marrow were pre-incubated in quadruplicate in 24-well plates in lml medium (StemSpan) with 15% FCS, SCF (20 ng/ml) and GM-CSF (2,5 ng/ml) for 6 to 7 days at 37° C. and 5.5% CO₂. Then compounds (0.1.1 or 10 μM in DMSO) with or without G-CSF (0.25 ng/ml; Neupogen®) were added and incubated for another 6 to 7 days. The number of the early myelopoietic CD15⁺/CD11b− cells and the number of the late myelopoietic CD15⁺/CD11b+ cells were determined by cell count (proliferation) and FACS (fluorescent associated cell sorting) analysis (differentiation) at day 13-14.

Megakaryoid CD34⁺ Liquid Culture

5×10³ CD34⁺ cells isolated from peripheral blood, cord blood or from bone marrow were incubated in quadruplicate 24-well plates in 1 ml serum-free medium with 2% BSA, SCF (20 ng/ml) and compounds (0.1,1 or 10 μM in DMSO) with or without TPO (0-10 ng/ml) for 12 to 13 days at 37° C. and 5% CO₂. The number of the megakaryoid CD41⁺ cells (scatter profile) were determined by FACS analysis. Megakaryocytes will be examined by microscope if necessary.

In Vivo Testing of Compounds/Target Validation

Erythropoiesis/Anemia

Compounds which have demonstrated effects on the drug target in vitro have been administered to normal or anemic animals orally or parenterally. In most cases, mice were used for compound testing. In some cases, other species, e.g. rats, hamsters or guinea pigs have been used in addition. Usually, repeated dosage is required for detection of changes in peripheral blood parameters. During the dosage period and up to five days after the last administration blood samples were drawn for analysis of red and white blood cell counts as well as platelet counts using an automated blood analyzer. In addition, erythropoiesis was assessed by manual hematocrit and reticulocyte count determination. For specific analysis of leukocyte differentiation fluorescent associated cell sorting (FACS) was used.

Myelopoiesis and Thrombocytopoiesis

Myelopoiesis

Immunocompetent Balb/c mice were treated with compounds at different doses (based on pharmacokinetic data) once/day or bid per-orally or parenterally for up to 4 days. The WBC (white blood cells count) and the neutrophil count were monitored by FACS (CD11b⁺; scatter properties).

Immunocompromised Balb/c were generated by intravenous treatment with 5-FU (100 mg/kg ip). 24 hours later the mice were treated with the test compound at different doses (based on pharmacokinetic data) once/day or bid per-orally or parenterally for up to 7 to 13 days. Peripheral blood counts (WBC, RBC, PLT) have been determined after retroorbital plexus puncture at days 5, 7, 11 and 14. For more detailed investigations the development of cellularity of femural bone marrow and spleen were investigated by FACS analysis. The expression of specific differentiation markers on stem and progenitor cells (e.g. CD34, CD33, CD38, CD11b) and scatter properties were investigated.

Thrombocytopoiesis

Thrombopoietic compounds at different doses (based on pharmacokinetic data) were administered orally or parenterally following chemotherapy (Carboplatin, 100 mg/kg ip) immunocompromised mice. After repeated administration (once/day or bid for five to seven days) peripheral blood platelets (automated blood analyzer) have been determined after retroorbital plexus puncture at day 5, 7, 11, and 14.

### EXAMPLE 13

Proliferation Inhibition Assay: Antisense Oligonucleotides Suppress the Growth of Cancer Cell Lines

The cell line used for testing is the human colon cancer cell line HCT116. Cells are cultured in RPMI-1640 with 10-15% fetal calf serum at a concentration of 10,000 cells per milliliter in a volume of 0.5 ml and kept at 37° C. in a 95% air/5% CO2 atmosphere.

Phosphorothioate oligoribonucleotides are synthesized on an Applied Biosystems Model 380B DNA synthesizer using phosphoroamidite chemistry. A sequence of 24 bases complementary to the nucleotides at position 1 to 24 of SEQ ID NO: 1 is used as the test oligonucleotide. As a control, another (random) sequence is used: 5′-TCA ACT GAC TAG ATG TAC ATG GAC-3′ (SEQ ID NO: 6). Following assembly and deprotection, oligonucleotides are ethanol-precipitated twice, dried, and suspended in phosphate buffered saline at the desired concentration. Purity of the oligonucleotides is tested by capillary gel electrophoresis and ion exchange HPLC. The purified oligonucleotides are added to the culture medium at a concentration of 10 μM once per day for seven days.

The addition of the test oligonucleotide for seven days results in significantly reduced expression of human hematopoietin receptor-like protein as determined by Western blotting. This effect is not observed with the control oligonucleotide. After 3 to 7 days, the number of cells in the cultures is counted using an automatic cell counter. The number of cells in cultures treated with the test oligonucleotide (expressed as 100%) is compared with the number of cells in cultures treated with the control oligonucleotide. The number of cells in cultures treated with the test oligonucleotide is not more than 30% of control, indicating that the inhibition of human hematopoietin receptor-like protein has an anti-proliferative effect on cancer cells.

### EXAMPLE 14

Cancer: In Vivo Testing of Compounds/Target Validation

Acute Mechanistic Assays

Reduction in Mitogenic Plasma Hormone Levels

This non-tumor assay measures the ability of a compound to reduce either the endogenous level of a circulating hormone or the level of hormone produced in response to a biologic stimulus. Rodents are administered test compound (p.o., i.p., i.v., i.m., or s.c.). At a predetermined time after administration of test compound, blood plasma is collected. Plasma is assayed for levels of the hormone of interest. If the normal circulating levels of the hormone are too low and/or variable to provide consistent results, the level of the hormone may be elevated by a pre-treatment with a biologic stimulus (i.e., LHRH may be injected i.m. into mice at a dosage of 30 ng/mouse to induce a burst of testosterone synthesis). The timing of plasma collection would be adjusted to coincide with the peak of the induced hormone response. Compound effects are compared to a vehicle-treated control group. An F-test is preformed to determine if the variance is equal or unequal followed by a Student's t-test. Significance is p value ≦0.05 compared to the vehicle control group.

Hollow Fiber Mechanism of Action Assay

Hollow fibers are prepared with desired cell line(s) and implanted intraperitoneally and/or subcutaneously in rodents. Compounds are administered p.o., i.p., i.v., i.m., or s.c. Fibers are harvested in accordance with specific readout assay protocol, these may include assays for gene expression (bDNA, PCR, or Taqman), or a specific biochemical activity (i.e., cAMP levels. Results are analyzed by Student's t-test or Rank Sum test after the variance between groups is compared by an F-test, with significance at p≦0.05 as compared to the vehicle control group.

Subacute Functional In Vivo Assays

Reduction in Mass of Hormone Dependent Tissues

This is another non-tumor assay that measures the ability of a compound to reduce the mass of a hormone dependent tissue (i.e., seminal vesicles in males and uteri in females). Rodents are administered test compound (p.o., i.p., i.v., i.m., or s.c.) according to a predetermined schedule and for a predetermined duration (i.e., 1 week). At termination of the study, animals are weighed, the target organ is excised, any fluid is expressed, and the weight of the organ is recorded. Blood plasma may also be collected. Plasma may be assayed for levels of a hormone of interest or for levels of test agent. Organ weights may be directly compared or they may be normalized for the body weight of the animal. Compound effects are compared to a vehicle-treated control group. An F-test is preformed to determine if the variance is equal or unequal followed by a Student's t-test. Significance is p value ≦0.05 compared to the vehicle control group.

Hollow Fiber Proliferation Assay

Hollow fibers are prepared with desired cell line(s) and implanted intraperitoneally and/or subcutaneously in rodents. Compounds are administered p.o., i.p., i.v., i.m., or s.c. Fibers are harvested in accordance with specific readout assay protocol. Cell proliferation is determined by measuring a marker of cell number (i.e., MTT or LDH). The cell number and change in cell number from the starting inoculum are analyzed by Student's t-test or Rank Sum test after the variance between groups is compared by an F-test, with significance at p≦0.05 as compared to the vehicle control group.

Anti-Angiogenesis Models

Corneal Angiogenesis

Hydron pellets with or without growth factors or cells are implanted irito a micropocket surgically created in the rodent cornea. Compound administration may be systemic or local (compound mixed with growth factors in the hydron pellet). Corneas are harvested at 7 days post implantation immediately following intracardiac infusion of colloidal carbon and are fixed in 10% formalin. Readout is qualitative scoring and/or image analysis. Qualitative scores are compared by Rank Sum test. Image analysis data is evaluated by measuring the area of neovascularization (in pixels) and group averages are compared by Student's t-test (2 tail). Significance is p≦0.05 as compared to the growth factor or cells only group.

Matrigel Angiogenesis

Matrigel, containing cells or growth factors, is injected subcutaneously. Compounds are administered p.o., i.p., i.v., i.m., or s.c. Matrigel plugs are harvested at predetermined time point(s) and prepared for readout. Readout is an ELISA-based assay for hemoglobin concentration and/or histological examination (i.e. vessel count, special staining for endothelial surface markers: CD31, factor-8). Readouts are analyzed by Student's t-test, after the variance between groups is compared by an F-test, with significance determined at p≦0.05 as compared to the vehicle control group.

Primary Antitumor Efficacy

Early Therapy Models

Subcutaneous Tumor

Tumor cells or fragments are implanted subcutaneously on Day 0. Vehicle and/or compounds are administered p.o., i.p., i.v., i.m., or s.c. according to a predetermined schedule starting at a time, usually on Day 1, prior to the ability to measure the tumor burden. Body weights and tumor measurements are recorded 2-3 times weekly. Mean net body and tumor weights are calculated for each data collection day. Anti-tumor efficacy may be initially determined by comparing the size of treated (T) and control (C) tumors on a given day by a Student's t-test, after the variance between groups is compared by an F-test, with significance determined at p≦0.05. The experiment may also be continued past the end of dosing in which case tumor measurements would continue to be recorded to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p≦0.05.

Intraperitoneal/Intracranial Tumor Models

Tumor cells are injected intraperitoneally or intracranially on Day 0. Compounds are administered p.o., i.p., i.v., i.m., or s.c. according to a predetermined schedule starting on Day 1. Observations of morbidity and/or mortality are recorded twice daily. Body weights are measured and recorded twice weekly. Morbidity/mortality data is expressed in terms of the median time of survival and the number of long-term survivors is indicated separately. Survival times are used to generate Kaplan-Meier curves. Significance is p≦0.05 by a log-rank test compared to the control group in the experiment.

Established Disease Model

Tumor cells or fragments are implanted subcutaneously and grown to the desired size for treatment to begin. Once at the predetermined size range, mice are randomized into treatment groups. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group. Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value ≦0.05 compared to the vehicle control group.

Orthotopic Disease Models

Mammary Fat Pad Assay

Tumor cells or fragments, of mammary adenocarcinoma origin, are implanted directly into a surgically exposed and reflected mammary fat pad in rodents. The fat pad is placed back in its original position and the surgical site is closed. Hormones may also be administered to the rodents to support the growth of the tumors. Compounds are administered p.o., i.p., i.v., i.m., or s.c. according to a predetermined schedule. Tumor and body weights are measured and recorded 2-3 times weekly. Mean tumor weights of all groups over days post inoculation are graphed for comparison. An F-test is preformed to determine if the variance is equal or unequal followed by a Student's t-test to compare tumor sizes in the treated and control groups at the end of treatment. Significance is p≦0.05 as compared to the control group.

Tumor measurements may be recorded after dosing has stopped to monitor tumor growth delay. Tumor growth delays are expressed as the difference in the median time for the treated and control groups to attain a predetermined size divided by the median time for the control group to attain that size. Growth delays are compared by generating Kaplan-Meier curves from the times for individual tumors to attain the evaluation size. Significance is p value ≦0.05 compared to the vehicle control group. In addition, this model provides an opportunity to increase the rate of spontaneous metastasis of this type of tumor. Metastasis can be assessed at termination of the study by counting the number of visible foci per target organ, or measuring the target organ weight. The means of these endpoints are compared by Student's t-test after conducting an F-test, with significance determined at p≦0.05 compared to the control group in the experiment.

Intraprostatic Assay

Tumor cells or fragments, of prostatic adenocarcinoma origin, are implanted directly into a surgically exposed dorsal lobe of the prostate in rodents. The prostate is externalized through an abdominal incision so that the tumor can be implanted specifically in the dorsal lobe while verifying that the implant does not enter the seminal vesicles. The successfully inoculated prostate is replaced in the abdomen and the incisions through the abdomen and skin are closed. Hormones may also be administered to the rodents to support the growth of the tumors. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the lungs), or measuring the target organ weight (i.e., the regional lymph nodes). The means of these endpoints are compared by Student's t-test after conducting an F-test, with significance determined at p≦0.05 compared to the control group in the experiment.

Intrabronchial Assay

Tumor cells of pulmonary origin may be implanted intrabronchially by making an incision through the skin and exposing the trachea. The trachea is pierced with the beveled end of a 25-gauge needle and the tumor cells are inoculated into the main bronchus using a flat-ended 27-gauge needle with a 90° bend. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the contralateral lung), or measuring the target organ weight. The means of these endpoints are compared by Student's t-test after conducting an F-test, with significance determined at p≦0.05 compared to the control group in the experiment.

Intracecal Assay

Tumor cells of gastrointestinal origin may be implanted intracecally by making an abdominal incision through the skin and externalizing the intestine. Tumor cells are inoculated into the cecal wall without penetrating the lumen of the intestine using a 27 or 30 gauge needle. Compounds are administered p.o., i.p., i.v., i.m., or s.c. according to a predetermined schedule. Body weights are measured and recorded 2-3 times weekly. At a predetermined time, the experiment is terminated and the animal is dissected. The size of the primary tumor is measured in three dimensions using either a caliper or an ocular micrometer attached to a dissecting scope. An F-test is preformed to determine if the variance is equal or unequal followed by a Student's t-test to compare tumor sizes in the treated and control groups at the end of treatment. Significance is p≦0.05 as compared to the control group. This model provides an opportunity to increase the rate of spontaneous metastasis of this type of tumor. Metastasis can be assessed at termination of the study by counting the number of visible foci per target organ (i.e., the liver), or measuring the target organ weight. The means of these endpoints are compared by Student's t-test after conducting an F-test, with significance determined at p≦0.05 compared to the control group in the experiment.

Secondary (Metastatic) Antitumor Efficacy

Spontaneous Metastasis

Tumor cells are inoculated s.c. and the tumors allowed to grow to a predetermined range for spontaneous metastasis studies to the lung or liver. These primary tumors are then excised. Compounds are administered p.o., i.p., i.v., i.m., or s.c. according to a predetermined schedule which may include the period leading up to the excision of the primary tumor to evaluate therapies directed at inhibiting the early stages of tumor metastasis. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope, and the mean target organ weights are compared by Student's t-test after conducting an F-test, with significance determined at p≦0.05 compared to the control group in the experiment for both of these endpoints.

Forced Metastasis

Tumor cells are injected into the tail vein, portal vein, or the left ventricle of the heart in experimental (forced) lung, liver, and bone metastasis studies, respectively. Compounds are administered p.o., i.p., i.v., i.m., or s.c. according to a predetermined schedule. Observations of morbidity and/or mortality are recorded daily. Body weights are measured and recorded twice weekly. Potential endpoints include survival time, numbers of visible foci per target organ, or target organ weight. When survival time is used as the endpoint the other values are not determined. Survival data is used to generate Kaplan-Meier curves. Significance is p≦0.05 by a log-rank test compared to the control group in the experiment. The mean number of visible tumor foci, as determined under a dissecting microscope, and the mean target organ weights are compared by Student's t-test after conducting an F-test, with significance at p≦0.05 compared to the vehicle control group in the experiment for both endpoints.

Expression Profiling

Total cellular RNA was isolated from cells by one of two standard methods: 1) guanidine isothiocyanate/cesium chloride density gradient centrifugation [Kellogg et al. (1990)]; or with the Tri-Reagent protocol according to the manufacturer's specifications (Molecular Research Center, Inc., Cincinatti, Ohio). Total RNA prepared by the Tri-reagent protocol was treated with DNAse I to remove genomic DNA contamination.

For relative quantitation of the mRNA distribution, total RNA from each cell or tissue source was first reverse transcribed. Eighty-five μg of total RNA was reverse transcribed using 1 μmole random hexamer primers, 0.5 mM each of dATP, dCTP, dGTP and dTTP (Qiagen, Hilden, Germany) and 3000 U RnaseQut (Invitrogen, Groningen, Netherlands) in a final volume of 680 μl. The first strand synthesis buffer and Omniscript reverse transcriptase (2 u/μl) were obtained from (Qiagen, Hilden, Germany). The reaction was incubated at 37° C. for 90 minutes and cooled on ice. The volume was adjusted to 6800 μl with water, yielding a final concentration of 12.5 ng/μl of starting RNA.

For relative quantitation of the distribution of mRNA in cells and tissues the Perkin Elmer ABI Prism R™ 7700 Sequence Detection system or Biorad iCycler was used according to the manufacturer's specifications and protocols. PCR reactions were set up to quantitate expression of the test gene and the housekeeping genes HPRT (hypoxanthine phosphoribosyltransferase), GAPDH (glyceraldehyde-3-phosphate dehydrogenase), β-actin, and others. Forward and reverse primers and probes were designed using the Perkin Elmer ABI Primer Express™ software and were synthesized by TibMolBiol (Berlin, Germany). The forward primer sequence was: Primer1 tctcagtctccccaggtgat (SEQ ID NO: 7). The reverse primer sequence was Primer2 gggctacaagatcaggagca (SEQ ID NO: 8). Probel catgtggcctcagaagccagagct (SEQ ID NO: 9), labeled with FAM (carboxyfluorescein succinimidyl ester) as the reporter dye and TAMRA (carboxytetramethylrhodamine) as the quencher, was used as a probe. The following reagents were prepared in a total of 25 μl: 1×TaqMan buffer A, 5.5 mM MgCl₂, 200 nM of dATP, dCTP, dGTP, and dUTP, 0.025 U/μl AmpliTaq Gold™, 0.01 U/μl AmpErase, and Probe1 catgtggcctcagaagccagagct, forward and reverse primers each at 200 nM, 200 nM , FAM/TAMRA-labeled probe, and 5 μl of template cDNA. Thermal cycling parameters were 2 min at 50° C., followed by 10 min at 95° C., followed by 40 cycles of melting at 95° C. for 15 sec and annealing/extending at 60° C. for 1 min.

Calculation of Corrected CT Values

The CT (threshold cycle) value is calculated as described in the “Quantitative determination of nucleic acids” section. The CF-value (factor for threshold cycle correction) is calculated as follows:
1. PCR reactions were set up to quantitate the housekeeping genes (HKG) for each cDNA sample.
2. CT_{HKG}-values (threshold cycle for housekeeping gene) were calculated as described in the “Quantitative determination of nucleic acids” section.
3. CT_{HKG}-mean values (CT mean value of all HKG tested on one cDNAs) of all HKG for each cDNA are calculated (n=number of HKG):
CT_{HKG-n}-mean value=(CT_{HKG1}-value+CT_{HKG2}-value+. . . . +CT_{HKG-n}-value)/n
4. CTₚₐₙₙₑₗmean value (CT mean value of all HKG in all tested cDNAs)=(CT_{HKG1}-mean value+CT_{HKG2}-mean value+. . . +CT_{HKG-y}-mean value)/y (y=number of cDNAs)
5. CF_{cDNA-n} (correction factor for cDNA n)=CTₚₐₙₙₑₗ-mean value−CT_{HKG-n}-mean value
6. CT_{cDNA-n} (CT value of the tested gene for the cDNA n)+CF_{cDNA-n}, (correction factor for cDNA n)=CT_{cor-DNA-n} (corrected CT value for a gene on cDNA n)
   Calculation of Relative Expression

Definition: highest CT_{cor-cDNA-n}≠40 is defined as CT_{cor-cDNA[high]}

Relative Expression=2^{(CTcor-cDNA[high]·CTcor-cDNA-n)}

The following tissues were tested: postcentral gyrus, aorta, heart ventricle (left), dorsal root ganglia, artery, retina, HUVEC cells, Alzheimer brain frontal lobe, cerebral meninges, breast, liver cirrhosis, esophagus, lung tumor, thrombocytes, neuroblastoma IMR32 cells, cerebellum (left), lymph node, rectum, penis, cerebellum (right), vein, bone marrow CD15⁺ cells, cerebral cortex, aorta sclerotic, Alzheimer brain, occipital lobe, ileum, breast tumor, heart atrium (left), cord blood CD71⁺ cells, lung COPD, vermis cerebelli, tonsilla cerebelli, colon tumor, ileum chronic inflammation, corpus callosum, fetal lung, frontal lobe, interventricular septum, hippocampus, erythrocytes, bone marrow CD34⁺ cells, HEP G2 cells, thyroid, coronary artery sclerotic, bone marrow CD71⁺ cells, cerebral peduncles, skeletal muscle, neuroblastoma SHSY cells, fetal kidney, spleen liver cirrhosis, HEK 293 cells, pericardium, skin, Alzheimer cerebral cortex, heart atrium (right), coronary artery smooth muscle primary cells, stomach, ovary tumor, adipose, ovary tumor, small intestine, temporal lobe, cerebellum, pons, bladder, Jurkat (T-cells), precentral gyrus, cervix, substantia nigra, pancreas liver cirrhosis, thalamus, fetal aorta, prostate, fetal heart, MDA MB 231 cells (breast tumor), testis, parietal lobe, colon, stomach tumor, leukocytes (peripheral blood), stomach tumor, adrenal gland, trachea, neuroblastoma SK-N-MC cells, prostate BPH, salivary gland, spinal cord, liver tumor, uterus, spleen, thyroid tumor, pancreas, kidney tumor, bone marrow, brain, heart, ileum tumor, uterus tumor, esophagus tumor, mammary gland, kidney, thymus, liver, placenta, coronary Artery, fetal liver, fetal lung fibroblast cells, HeLa cells (cervix tumor), fetal brain, lung.

The results are shown in Table 1.

| Tissue | Relative Expression |
|---|---|
| postcentral gyrus | 9027 |
| Aorta | 6295 |
| heart ventricle (left) | 3984 |
| dorsal root ganglia | 3566 |
| Artery | 3040 |
| Retina | 3019 |
| HUVEC cells | 2798 |
| Alzheimer brain frontal lobe | 2740 |
| cerebral meninges | 2574 |
| Breast | 2402 |
| liver cirrhosis | 2210 |
| esophagus | 2077 |
| lung tumor | 2048 |
| thrombocytes | 2034 |
| neuroblastoma IMR32 cells | 2006 |
| cerebellum (left) | 1808 |
| lymph node | 1783 |
| rectum | 1652 |
| Penis | 1563 |
| cerebellum (right) | 1342 |
| Vein | 1305 |
| bone marrow CD15⁺ cells | 1261 |
| cerebral cortex | 1075 |
| aorta sclerotic | 1075 |
| Alzheimer brain | 1038 |
| occipital lobe | 1031 |
| Ileum | 891 |
| Breast tumor | 826 |
| heart atrium (left) | 809 |
| cord blood CD71⁺ cells | 653 |
| lung COPD | 648 |
| vermis cerebelli | 644 |
| tonsilla cerebelli | 639 |
| colon tumor | 592 |
| Ileum chronic inflammation | 576 |
| corpus callosum | 560 |
| fetal lung | 516 |
| frontal lobe | 484 |
| interventricular septum | 410 |
| hippocampus | 407 |
| erythrocytes | 391 |
| Bone marrow CD34⁺ cells | 377 |
| HEP G2 cells | 377 |
| thyroid | 377 |
| coronary artery sclerotic | 372 |
| Bone marrow CD71⁺ cells | 355 |
| cerebral peduncles | 317 |
| skeletal muscle | 294 |
| neuroblastoma SH5Y cells | 280 |
| fetal kidney | 267 |
| spleen liver cirrhosis | 260 |
| HEK 293 cells | 256 |
| pericardium | 231 |
| Skin | 229 |
| Alzheimer cerebral cortex | 223 |
| Heart atrium (right) | 218 |
| coronary artery smooth | 201 |
| muscle primary cells | |
| stomach | 187 |
| ovary tumor | 182 |
| adipose | 182 |
| ovary tumor | 182 |
| small intestine | 165 |
| temporal lobe | 148 |
| cerebellum | 146 |
| pons | 144 |
| bladder | 137 |
| Jurkat (T-cells) | 130 |
| precentral gyrus | 126 |
| cervix | 120 |
| substantia nigra | 119 |
| pancreas liver cirrhosis | 115 |
| thalamus | 112 |
| fetal aorta | 106 |
| prostate | 103 |
| fetal heart | 100 |
| MDA MB 231 cells (breast tumor) | 96 |
| testis | 92 |
| parietal lobe | 83 |
| colon | 73 |
| stomach tumor | 70 |
| leukocytes (peripheral blood) | 70 |
| stomach tumor | 70 |
| adrenal gland | 64 |
| trachea | 62 |
| neuroblastoma SK-N-MC cells | 49 |
| prostate BPH | 48 |
| salivary gland | 46 |
| spinal cord | 40 |
| liver tumor | 35 |
| uterus | 31 |
| spleen | 26 |
| thyroid tumor | 23 |
| pancreas | 22 |
| kidney tumor | 20 |
| bone marrow | 17 |
| brain | 15 |
| heart | 15 |
| ileum tumor | 13 |
| uterus tumor | 11 |
| esophagus tumor | 11 |
| mammary gland | 8 |
| kidney | 7 |
| thymus | 5 |
| liver | 4 |
| placenta | 4 |
| coronary Artery | 2 |
| fetal liver | 2 |
| fetal lung fibroblast cells | 1 |
| HeLa cells (cervix tumor) | 1 |
| fetal brain | 1 |
| lung | 0 |

### REFERENCES

1. Human hematopoietic growth factors: old lessons and new perspectives, Dempke W, Von Poblozki A, Grothey A, Schmoll H J, Anticancer Res 2000 November-December; 20(6D):5155-64.
2. Hematopoietic growth factors in cancer chemotherapy, Crawford J, Foote M, Morstyn G, Cancer Chemother Biol Response Modif 1999;18:250-67.
3. Transcriptional regulation of vascular development, Oettgen P, Circ Res 2001 Aug. 31; 89(5):380-8.
4. The dynamics of bone marrow stromal cells in the proliferation of multipotent hematopoietic progenitors by substance P: an understanding of the effects of a neurotransmitter on the differentiating hematopoietic stem cell, Rameshwar P, Zhu G, Donnelly RJ, Qian J, Ge H, Goldstein K R, Denny T N, Gascon P, J Neuroimmunol 2001 Dec. 3; 121(1-2):22-31.

1-17. (canceled)

18. An isolated and purified protein comprising an amino acid sequence selected from the group consisting of the amino acid sequences shown in SEQ ID NOS:2 and 5.

19. An isolated and purified protein comprising an amino acid sequence which is at least 90% identical to the amino acid sequence shown of claim 18 and which has a receptor activity.

20. A purified preparation of antibodies which specifically bind to the protein of claim 18.

21. The preparation of claim 20 wherein the antibodies are polyclonal.

22. The preparation of claim 20 wherein the antibodies are monoclonal.

23. The preparation of claim 20 wherein the antibodies are single-chain antibodies.

24. The preparation of claim 20 wherein the antibodies are Fab, F(ab′)₂, or Fv fragments.

25. An isolated and purified polynucleotide which encodes the protein of claim 18.

26. The polynucleotide of claim 25 which comprises a nucleotide sequence selected from the group consisting of the nucleotide sequence shown in SEQ ID NOS: 1, 3 and 4.

27. The polynucleotide of claim 25 which is a cDNA.

28. An isolated and purified single-stranded polynucleotide comprising at least 8 contiguous nucleotides of a coding sequence or a complement of the coding sequence for the protein of claim 18.

29. The polynucleotide of claim 28 wherein the coding sequence comprises a nucleotide sequence selected from the group consisting of the nucleotide sequence shown in SEQ ID NOS:1, 3 and 4.

30. An expression construct, comprising;
a coding sequence for the protein of claim 18; and
a promoter which is located upstream from the coding sequence and which controls expression of the coding sequence.

31. The expression construct of claim 30 wherein the coding sequence comprises a nucleotide sequence selected from the group consisting of the nucleotide sequence shown in SEQ ID NOS:1, 3 and 4.

32. A host cell comprising the expression construct of claim 30.

33. The host cell of claim 32 which is prokaryotic.

34. The host cell of claim 32 which is eukaryotic.

35. A method of producing a protein, comprising the steps of:
culturing the host cell of claim 32 under conditions whereby the protein is expressed; and
recovering the protein.

36. A method of detecting an expression product of a gene encoding the protein of claim 18, comprising the steps of:
contacting a test sample with a reagent that specifically binds to an expression product of a nucleotide sequence selected from the group consisting of the nucleotide sequence shown in SEQ ID NOS:1, 3 and 4;
assaying the test sample to detect binding between the reagent and the expression product; and
identifying the test sample as containing the expression product if binding between the reagent and the expression product is detected.

37. The method of claim 36 wherein the expression product is a protein.

38. The method of claim 36 wherein the reagent is an antibody.

39. The method of claim 36 wherein the cell is cultured in vitro and wherein the test sample is culture medium.

40. The method of claim 36 wherein the expression product is an mRNA molecule.

41. The method of claim 40 wherein the reagent is an antisense oligonucleotide.

42. A method of treating, comprising the step of:
administering to a patient having a disorder selected from the group consisting of a CNS disorder, COPD, a cardiovascular disorder, a liver disorder, cancer, asthma, and a hematological disorder an effective amount of the antibody of claim 20 whereby symptoms of the disorder are reduced.

43. A method of treating comprising the step of:
administering to a patient having a disorder selected from the group consisting of a CNS disorder, COPD, a cardiovascular disorder, a liver disorder, cancer, asthma, and a hematological disorder an effective amount of an antisense oligonucleotide that regulates expression of a gene encoding the protein of claim 18.

44. A method of screening for candidate therapeutic agents, comprising the steps of:
contacting the protein of claim 18 with a test compound;
assaying for binding between the protein and the test compound; and
identifying a test compound that binds to the protein as a candidate therapeutic agent that may be useful for treating a disorder selected from the group consisting of a CNS disorder, COPD, a cardiovascular disorder, a liver disorder, cancer, asthma, and a hematological disorder.

45. The method of claim 44 wherein either the test compound or the protein comprises a detectable label.

46. The method of claim 44 wherein either the test compound or the protein is bound to a solid support.

47. A method of screening for candidate therapeutic agents, comprising the steps of:
assaying for expression of a polynucleotide encoding the protein of claim 18 in the presence and absence of a test compound; and
identifying a test compound that regulates the expression as a candidate therapeutic agent that may be useful for treating a disorder selected from the group consisting of a CNS disorder, COPD, a cardiovascular disorder, a liver disorder, cancer, asthma, and a hematological disorder.

48. The method of claim 47 wherein the step of contacting is in a cell.

49. The method of claim 47 wherein the step of contacting is in a cell-free in vitro translation system.

50. A pharmaceutical composition comprising
a therapeutic reagent selected from the group consisting of the preparation of claim 20, an antisense oligonucleotide which binds to an expression product of a gene which encodes the protein of claim 18, the protein of claim 18, and the polynucleotide of claim 25; and
a pharmaceutically acceptable carrier.

51. The pharmaceutical composition of claim 50 wherein the polynucleotide comprises a nucleotide sequence selected from the group consisting of the nucleotide sequence shown in SEQ ID NOS:1, 3 and 4.
